(19) United States
(12) Patent Application Publication
(10) Pub. No.: US 2023/0140635 A1
(43) Pub. Date: May 4, 2023

(54) 3-(2-(Aminoethyl)-Indol-4-ol Derivatives, Methods of Preparation Thereof, and the Use as 5-HT2 Receptor Modulators

(71) Applicants: Bright Minds Biosciences Inc., Vancouver (CA); The Medical College of Wisconsin, Inc., Milwaukee, WI (US)

(72) Inventors: Alan Kozikowski, Chicago, IL (US); Gideon Shapiro, Gainesville, FL (US); Werner Tueckmantel, Fitchburg, WI (US); John McCorvy, Milwaukee, WI (US)

(21) Appl. No.: 17/911,022

(22) PCT Filed: Mar. 12, 2021
(86) PCT No.: PCT/CA2021/050336

§ 371 Date: Sep. 12, 2022

Related U.S. Application Data
(60) Provisional application No. 62/988,826, filed on Mar. 12, 2020.

(51) Int. Cl. C07D 209/32

(52) U.S. Cl. CPC C07D 209/32

(57) ABSTRACT

A compound of Formula I, which possesses 5-HT_{2A} and/or 5-HT_{2C} selective receptor activity, but lacks at lease some of the undesirable characteristics of 5-HT2B-agonist related activities, is disclosed. Methods of preparing said compounds are also described. The compound of Formula I may be useful in the treatment of depression, alcoholism, tobacco and cocaine addiction, inflammation, cluster headache, PTSD, seizure disorders and other CNS disorders.

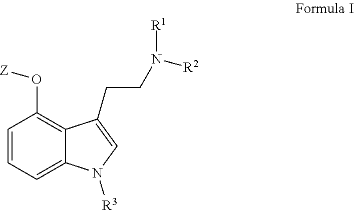

### TECHNICAL FIELD:

Described in this disclosure are novel indole compounds, and the methods of preparing the same. Also described in this disclosure are the uses of the same as selective agents at serotonin receptors. Also described in this disclosure are compounds that modulate 5-HT2 receptors.

### BACKGROUND:

Psilocybin is a naturally occurring psychedelic prodrug compound produced by more than 200 species of mushrooms which are collectively known as psilocybin mushrooms. As a prodrug, psilocybin is quickly metabolized by the body to generate the bioactive compound psilocin, which has mind-altering effects not unlike those produced by LSD, mescaline, and DMT. These effects include, inter alia, euphoria, visual and mental hallucinations, changes in perception, a distorted sense of time, and spiritual experiences, and can also include possible adverse reactions such as nausea and panic attacks.

While psilocybin and its therapeutic potential, along with that of other psychedelic drugs like LSD in psychiatry, was recognized and explored over 50 years ago by Hofmann and co-workers at Sandoz (see, for example, Hofmann, A., Troxler, F. U.S. Pat. No. 3,075,992; U.S. Pat. No. 3,078,214), subsequent investigation into the recreational use of psilocybin and related psychedelic drugs (e.g. LSD) was curtailed in the early 1970s. Since then, psilocybin remains classified as a scheduled drug of abuse in most countries by many national drug laws.

However, clinical investigations have recently led to increased awareness of the potential for psychedelic drugs and psilocybin in particular as breakthrough therapies to treat CNS diseases of unmet medical need. These diseases that may be addressed include both difficult to treat mental health disorders (Daniel J, Haberman M. Clinical potential of psilocybin as a treatment for mental health conditions. Ment. Health Clin. 2017, 7(1), 24-8) associated with significant morbidity such as treatment resistant depression (TRD), along with alcoholism and cocaine and tobacco addiction, as well as neurological disorders such as cluster headache which also has significant associated morbidity.

As a phosphate phenolic prodrug, psilocybin, once ingested is rapidly metabolized to the bioactive constituent psilocin, which then acts on serotonin receptors in the brain. The 5-hydroxytryptamine receptors (5-HT) receptors, or serotonin receptors, are a group of G protein-coupled receptors and ligand-gated ion channels found in both the central and peripheral nervous systems. They mediate both excitatory and inhibitory neurotransmission. The 5-HT receptors are activated by the neurotransmitter 5-hydroxytryptamine more commonly known as serotonin, which is the natural ligand.

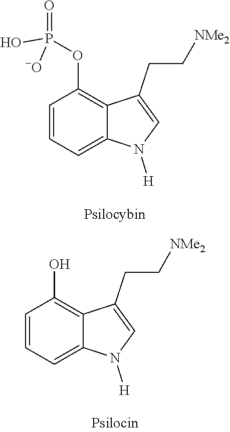

Other prodrug linkages are taught in the literature, such as Wiemer et al. Top Curr Chem. 2015; 360: 115-160 (incorporated herein by reference) and Mahato et al. Adv Drug Deliv Rev. 2011 Jul. 18; 63(8): 659-670 (incorporated herein by reference).

As a novel antidepressant, in a small clinical trial involving cancer patients treated with psilocybin, it was found that high-dose psilocybin produced large decreases in clinician- and self-rated measures of depressed mood and anxiety, along with increases in quality of life, life meaning, and optimism, and decreases in death anxiety. In a 6-month follow-up, these changes were sustained, with about 80% of participants continuing to show clinically significant decreases in depressed mood and anxiety.

In a proof-of-concept study for psilocybin for use in treating alcohol dependence, and that involved 10 patients with a diagnosis of alcohol dependence per the DSM-IV, a significant decrease in alcohol use post psilocybin administration among the patients was observed (Bogenschutz MP, et al., Psilocybin-assisted treatment for alcohol dependence: a proof-of-concept study. J. Psychopharmacol. 2015, 29(3), 289-99). In a study on the treatment of tobacco addiction with psilocybin, researchers found that eighty percent of participants showed biologically verified 7-day point-prevalence abstinence at 6-month follow up. The 12-month follow-up showed 67% of the participants were biologically verified as being abstinent. A follow-up 2.5 years after the target quit date showed 75% were abstinent (Johnson, M. W., and Griffiths, R. R. (2017) Potential Therapeutic Effects of Psilocybin. Neurotherapeutics 14, 734).

Pharmacologically, the bioactive constituent psilocin has been shown to act at a number of serotonin receptor subtypes from in vitro receptor binding and functional assays as summarized in Table 1 below, which is in line with its structural resemblance to serotonin (Geiger H. A., Wurst M. G., Daniels R. N., “DARK Classics in Chemical Neuroscience: Psilocybin,” ACS Chem. Neurosci. 2018, 9 (10), 2438-2447).

TABLE 1
Pharmacological Properties of Psilocin

| Binding Site | Kᵢ (nM) and EC₅₀ Values |
|---|---|
| SERT | 3801 |
| 5-HT1A | 567 |
| 5-HT1B | 219 |
| 5-HT1D | 36 |
| 5-HT2A | 107; EC₅₀ = 24 (43%) |
| 5-HT2B | 4.6; EC₅₀ = 58 (45%) |
| 5-HT2C | 97; EC₅₀ = 12 (45%) |
| 5-HT3 | >10000 |
| 5-HT5 | 84 |
| 5-HT6 | 57 |
| 5-HT7 | 3.5 |

EC₅₀ values for activation of PI hydrolysis in cells expressing human receptors relative to serotonin at 100%. Efficacy is provided in parentheses.

Psilocin exhibited no significant effect on dopamine receptors (unlike LSD) and appears to only act upon the noradrenergic system at very high dosages. The diverse pharmacological effects of particular relevance to therapeutic utility and limitations can be ascribed to psilocin's activation of 5-HT2A, 5-HT2B, and 5-HT2C receptors specifically as a functional agonist.

A major limitation to the therapeutic potential for psilocybin is a serious toxicological safety liability, namely cardiac valvulopathy, that can be anticipated from psilocin's potent agonist activity at 5-HT2B receptors. Thus, previous drugs with 5-HT2B receptor agonist activity have been found to have life threatening side effects such as cardiac valvulopathy (Rothman, R., Baumann, M., Savage, J., Rauser, L., McBride, A., Hufeisen, S., Roth, B. L. “Evidence for Possible Involvement of 5-HT2B Receptors in the Cardiac Valvulopathy Associated with Fenfluramine and other Serotonergic Medications” Circulation 2000, 102, 2836; Fitzgerald, L., Bum, T., Brown, B., Patterson, J., Corjay, M., Valentine, P., Sun, J-H., Link, J., Abbaszade, I., Hollis, J., Largent, B., Hartig, P., Hollis, G., Meunier, P., Robichaud, A., Robertson, D. “Possible Role of Valvular Serotonin 5-HT2B Receptors in the Cardiopathy Associated with Fenfluramine” Mol. Pharmacol. 2000, 57, 75) and pulmonary hypertension (Launay, J., Herve, P., Peoc'h, K., Toumois, C., Callebert, J., Nebigil, C., Etienne, N., Drouet, L., Humbert, M., Simonneau, G., Maroteaux, L. “Function of the Serotonin 5-Hydroxytryptamine 2B Receptor in Pulmonary Hypertension” Nature Med. 2002, 8, 1129).

The development of 5-HT2C receptor agonists acting in the brain has been focused on treating obesity (Bickerdike, M., Vickers, S., Dourish, C. “5-HT2C Receptor Modulation and the Treatment of Obesity” Diabetes Obes. Metab. 1999, 1, 207; Martin, J., Bös, M., Jenck, F., Moreau, J-L., Mutel, V., Sleight, A., Wichmann, J., Andrews, J., Berendsen, H., Broekkamp, C., Ruight, G., Köhler, C., van Delft, A. M. L. “ 5-HT2C Receptor Agonists: Pharmacological Characteristics and Therapeutic Potential” J. Pharm. Exp Ther. 1998, 286, 913). Locaserin, a selective 5-HT2C agonist over 5-HT2B, was shown to have no increased risk of cardiac valvulopathy in patients and was FDA approved for treating obesity (Shukla A P, Kumar R B, Aronne L J (2015). “Lorcaserin HCl for the treatment of obesity”. Expert Opinion on Pharmacotherapy. 16(16): 2531-8).

Sard et al. have described the synthesis and characterization of the 5-HT2 receptor activity of psilocin analogs (Sard H., Kumaran G., Morency C., Roth B. L., Toth B. A., He P., Shuster L. “SAR of psilocybin analogs: discovery of a selective 5-HT2C agonist” Bioorg. Med. Chem. Lett. 2005, 15(20), 4555-9; Sard et al., Indole Compounds Useful as Serotonin Selective Agents; WO2006/047302; Sard et al., Indole Compounds and Methods of Use Thereof, US2009/033822). The aim of these studies to identify 5-HT2C selective agonists was realized, and psilocin analogs were identified with potent 5-HT2C agonist functional activity that were devoid of 5-HT2B agonist functional activity. Selected analogs with this profile were also tested in an obsessive compulsive disorder (OCD) mouse behavior model versus psilocybin and psilocin as active reference compounds (Sard H., Kumaran G., Morency C., Roth B. L., Toth B. A., He P., Shuster L. “SAR of psilocybin analogs: discovery of a selective 5-HT2C agonist” Bioorg. Med. Chem. Lett. 2005, 15(20), 4555-9). In this report, the 1-methyl analog compound of psilocin, compound 1, was found to have high functional agonist selectivity for activation of phosphoinisitol hydrolysis on human 5-HT2C receptors vs the 5-HT2A and 5-HT2B receptors. For the human 5-HT2C receptor compound 1 was reported to have 12 nM potency with ca. 45% functional efficacy response vs serotonin (set at 100%). At the human 5-HT2A receptor compound A was reported to have a 633 nM potency with ca. 30% functional efficacy response vs serotonin. Compound 1 was reported to be inactive as an agonist but a potent antagonist (38 nM) at 5-HT2B receptors. Finally, efficacy in a mouse OCD model demonstrated for compound 1 was ascribed to 5-HT2C receptor agonist activity. Compound 1, was later found to be efficacious in the mouse head twitch assay model for 5-HT2A activity but with much lower potency relative to psilocin (Halberstadt, A. L., Koedood, L. Powell, S. B. and Geyer, M. A. “Differential contributions of serotonin receptors to the behavioral effects of indoleamine hallucinogens in mice” Journal of Psychopharmacology, 2011, 25, 1548-1561). Compound 6, the 1-butyl analog of psilocin was reported to a selective for 5-HT2C agonist but with much lower potency (ca. 600 nM) indicating dramatic loss of potency with increasing size of the group substitution at the 1-position indole nitrogen.

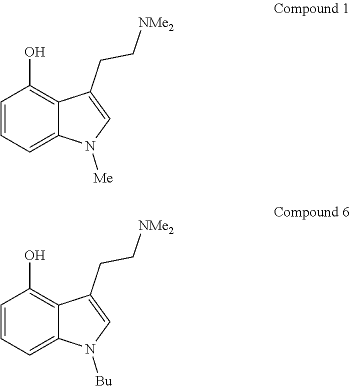

While psilocybin has recognized therapeutic potential for treating diverse CNS diseases and disorders including treatment-resistant depression (TRD), alcoholism, tobacco use, and cluster headache, there is an unmet need for safer drugs and analogs of psilocin that maintain 5-HT2A receptor agonist activity but that lack cardiotoxic 5-HT2B agonist activity.

Compounds with this selectivity profile for 5-HT2A over 5-HT2B are described as having broad potential to treat a variety of CNS diseases. However, because psilocybin does not fit this particular profile, its potential application as a drug therapy remains limited. It would be desirable to create psilocin analogs that have some of the same pharmacological properties as psilocin but lack its 5-HT2B agonist activity, which has been linked to undesirable side effects such as cardiac valvulopathy.

### SUMMARY

Described herein are (i) certain analogs that bear a substituent on the indole nitrogen and (ii) mimics of psilocybin and related prodrugs in which an ester, phosphate or urethane function is located at the 4-position of the indole ring.

Compounds disclosed herein are able to maintain 5-HT2A, 5-HT2C, or both 5-HT2A and 5-HT2C selective receptor agonist activity while lacking at least some of the undesirable characteristics of 5-HT2B-agonist related activities of psilocybin/psilocin. Compounds disclosed herein may be useful in the treatment of depression, alcoholism, tobacco and cocaine addiction, inflammation, cluster headache, PTSD, and other CNS disorders.

According to a part of the disclosure, there are chemical entities of Formula (I):

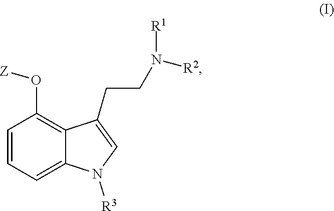

wherein R¹, R², R³, and Z are defined herein.

Chemical entities of Formula (I), and pharmaceutically acceptable compositions thereof, are expected to be useful for treating a variety of diseases and disorders associated with 5-HT2A receptor agonism. Such diseases and disorders include those described herein.

According to another part of the disclosure, there is a chemical entity of Formula I or a pharmaceutically acceptable salt thereof wherein: (i) R¹ is C₁-₆ alkyl, C₂-₆ alkenyl, C₂-₆ alkynyl, C₃-C₆ cycloalkyl, or CH₂(C₃-C₆ cycloalkyl), substituted benzyl, halobenzyl, C₁-C₆ alkyl benzyl, C₁-C₆ alkoxy benzyl or C₁-C₆ alkyl aryl; (ii) R² is H, C₁-C₆ alkyl, C₂-C₆ alkenyl, C₂-C₆ alkynyl, C₃-C₆ cycloalkyl, CH₂(C₃-C₆ cycloalkyl), substituted benzyl, halobenzyl, C₁-C₆ alkyl benzyl, C₁-C₆ alkoxy benzyl or C₁-C₆ alkyl aryl; and (iii) R³ is C₁-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl); and wherein: (A) if R¹ and R² are C₁ alkyl, then R³ is C₂-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl); (B) if R¹ and R³ are C₁ alkyl, then R² is H, C₂-C₆ alkyl, C₂-C₆ alkenyl, C₂-C₆ alkynyl, C₃-C₆ cycloalkyl, CH₂(C₃-C₆ cycloalkyl), or alkylaryl; (C) if R² and R³ are C1 alkyl, then R¹ is H, C₂-C₆ alkyl, C₂-C₆ alkenyl, C₂-C₆ alkynyl, C₃-C₆ cycloalkyl, or CH₂(C₃-C₆ cycloalkyl); and (D) if R¹ and R² are C₁alkyl, then R³ is C₁-C₃ alkyl, C₅-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl).

R¹ and R² may be joined to form a 4 membered heterocyclyl group, 5 membered heterocyclyl group, 6 membered heterocyclyl group, or 7 membered heterocyclyl group.

Alkyl substituents may comprise one or more fluorine atoms.

According to another part of the disclosure, there is a chemical entity of Formula I or a pharmaceutically acceptable salt thereof, wherein R¹ and R² are C₁ alkyl and R³ is ethyl.

According to another part of the disclosure, there is a chemical entity of Formula I or a pharmaceutically acceptable salt thereof, wherein R¹ and R² are C₁ alkyl and R³ is CH₂(cyclopropyl).

According to another part of the disclosure, there is a chemical entity of Formula I or a pharmaceutically acceptable salt thereof, wherein R¹ and R² are C₁ alkyl and R³ is cyclopropyl.

According to another part of the disclosure, there is a chemical entity of Formula I or a pharmaceutically acceptable salt thereof, wherein R¹ and R³ are C₁ alkyl and R² is CH₂(cyclopropyl).

According to another part of the disclosure, there is a chemical entity of Formula I or a pharmaceutically acceptable salt thereof, wherein R¹ and R² are each CH₃ and R³ is CD₃.

According to another part of the disclosure, there is a chemical entity of Formula I or a pharmaceutically acceptable salt thereof, wherein R¹ is C₁-C₆ alkoxy benzyl, R² is H, and R³ is C₁-C₆ alkyl.

According to another part of the disclosure, there is a chemical entity of Formula I or a pharmaceutically acceptable salt thereof, wherein R¹ is substituted benzyl, R² is H, and R³ is C₁-C₆ alkyl. The substituted benzyl may comprise one, two, or three substituents selected from the group consisting of: C₁-C₄ alkyl, C₁-C₄ alkoxy, halo, and any combination thereof.

According to another part of the disclosure, there is a method for treating the symptoms of any one of depression, alcoholism, tobacco and cocaine addiction, inflammation, cluster headache, PTSD, and other CNS disorders, in a subject comprising administering to said subject an effective amount of a chemical entity of Formula I or a pharmaceutically acceptable salt thereof.

According to another part of the disclosure, there is a use of a chemical entity of Formula I or a pharmaceutically acceptable salt thereof, wherein an effective amount of said compound is administered into a subject.

According to another part of the disclosure, there is a use of a chemical entity of Formula I or a pharmaceutically acceptable salt thereof to treat the symptoms of any one of depression, alcoholism, tobacco and cocaine addiction, inflammation, cluster headache, PTSD, seizure disorders, and other CNS disorders.

According to another part of the disclosure, there is a use of a chemical entity of Formula I or a pharmaceutically acceptable salt thereof in the manufacture of a medicament for the treatment of the symptoms of any one of depression, alcoholism, tobacco and cocaine addiction, inflammation, cluster headache, PTSD, seizure disorders, and other CNS disorders.

This summary does not necessarily describe the entire scope of all aspects of the disclosure. Other aspects, features and advantages will be apparent to those of ordinary skill in the art upon review of the following description of specific embodiments.

### DETAILED DESCRIPTION

In this disclosure, chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

As used herein, the term “about”, when used to describe a recited value, means within 5% of the recited value.

As used herein, the term “alkoxy”, used alone or as part of a larger moiety, refers to the groups —O -alkyl and —O-cycloalkyl.

As used herein, the term “alkyl”, used alone or as part of a larger moiety, means a substituted or unsubstituted, linear or branched, univalent hydrocarbon chain that is completely saturated. Unless otherwise specified, alkyl groups contain 1 to 7 carbon atoms (“C₁-C₇ alkyl”). In some embodiments, alkyl groups contain 1 to 6 carbon atoms (“C₁-C₆ alkyl”). In some embodiments, alkyl groups contain 1 to 5 carbon atoms (“C₁-C₅ alkyl”). In some embodiments, alkyl groups contain 1 to 4 carbon atoms (“C₁-C₄ alkyl”). In some embodiments, alkyl groups contain 3 to 7 carbon atoms (“C₃-C₇ alkyl”). Examples of saturated alkyl groups include methyl, ethyl, n-propyl, i-propyl, n-butyl, t-butyl, i-butyl, s-butyl, homologs and isomers of, for example, n-pentyl, n-hexyl, n-heptyl, n-octyl, and the like. The term “lower alkyl” refers to alkyl groups having 1 to 4 carbon atoms. Examples of lower alkyl groups include methyl, ethyl, n-propyl, ipropyl, n-butyl, s-butyl, i-butyl, t-butyl, and the like. A substituted alkyl group is one having at least one but no more than five substituents, and no more substituents than the number of hydrogen atoms in the unsubstituted group. In some embodiments, the substituents comprise hydrogen atoms. In some embodiments, the substituents comprise fluorine atoms. In some embodiments, the substituents comprise deuterium atoms. Examples of substituted alkyl groups include 2-hydroxyethyl, 2-methoxyethyl, CHF₂, CF₃, CH₂CHF₂ CH₂CF₃, CF₂CF₃, 4-fluorobutyl, CD₃ and the like. The term “alkenyl” refers to a substituted or unsubstituted, linear or branched, univalent hydrocarbon chain having at least two carbon atoms and at least one carbon-carbon double bond. Examples of alkenyl groups include allyl, vinyl, 2-propenyl, crotyl, 2-isopentenyl, 1,3-butadien-2-yl, 2,4-pentadien-1-yl, 1,4-pentadien-3-yl, and the like. The term “alkynyl” refers to a substituted or unsubstituted, linear or branched, univalent hydrocarbon chain having at least two carbon atoms and at least one carbon-carbon triple bond. Examples of alkynyl groups include ethynyl, 1- and 3-propynyl, 3-butyn-1-yl, and the like.

As used herein, the term “aryl”, used alone or as part of a larger moiety, e.g., “(aryl)alkyl”, refers to a univalent monocyclic or bicyclic carbocyclic aromatic ring system. Unless otherwise specified, aryl groups contain 6 or 10 ring members. Examples of aryl include phenyl, naphthyl, and the like. Aryl groups may be unsubstituted or may be substituted with one, two, or three groups selected independently from halogen, OH, C₁-C₆ alkoxy, and C₁-C₆ alkyl.

As used herein, the term “carrier” refers to a diluent, adjuvant, or excipient, with which a psilocybin analog described herein may be administered. Such pharmaceutical carriers can be liquids, such as water and oils, including those of petroleum, animal, vegetable or synthetic origin, such as peanut oil, soybean oil, mineral oil, sesame oil, and the like. The carriers can be saline, gum acacia, gelatin, starch paste, talc, keratin, colloidal silica, urea, and the like. In addition, auxiliary, stabilizing, thickening, lubricating, and coloring agents can be used. The pharmaceutically acceptable carriers are sterile.

As used herein, the term “chemical entity” refers to a compound having the indicated structure, whether in its “free” form (e.g., “free compound” or “free base” or “free acid” form, as applicable), or in a salt form, particularly a pharmaceutically acceptable salt form, and furthermore whether in solid state form or otherwise. In some embodiments, a solid state form is an amorphous (i.e., non-crystalline) form; in some embodiments, a solid state form is a crystalline form. In some embodiments, a crystalline form (e.g., a polymorph, pseudohydrate, or hydrate). Similarly, the term encompasses the compound whether provided in solid form or otherwise. Unless otherwise specified, all statements made herein regarding “compounds” apply to the associated chemical entities, as defined.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method or use functions. The term “consisting of” when used herein in connection with a composition, use or method, excludes the presence of additional elements and/or method steps.

As used herein, the term “cycloalkyl”, used alone or as part of a larger moiety, e.g., “(cycloalkyl)alkyl”, refers to a substituted or unsubstituted, univalent monocyclic hydrocarbon that is completely saturated or that contains one or more units of unsaturation, but which is not aromatic; or bicyclo[2.2.1]heptyl (also called norbornyl) or bicyclo[2.2.2]octyl. In some embodiments, cycloalkyl groups contain 3 to 8 ring carbon atoms (“C₃-C₈ cycloalkyl”). Examples of cycloalkyl include cyclopropyl, cyclobutyl, cyclopentyl, cyclohexyl, 1-cyclohexenyl, 3-cyclohexenyl, cycloheptyl, and the like, as well as bicyclo[2.2.1]heptyl and bicyclo[2.2.2]octyl. A substituted cycloalkyl group is one having at least one but no more than five substituents. In some embodiments, the substituents are fluorine atoms. Examples of substituted cycloalkyl groups include 4-hydroxycyclohexyl, 2-methoxycyclopentyl, 4,4-difluorocyclohexyl, and the like.

As used herein, the term “halogen” or “halo”, used alone or as part of a larger moiety, refers to fluoro, chloro, bromo or iodo.

As used herein, the term “heteroaryl”, used alone or as part of a larger moiety, e.g., “(heteroaryl)alkyl”, refers to a univalent monocyclic or bicyclic group having 5 to 10 ring atoms, preferably 5, 6, 9, or 10 ring atoms, having 6 or 10 π electrons shared in a cyclic array, and having, in addition to ring carbon atoms, from one to four ring heteroatoms. Examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolyl, indolizinyl, benzofuranyl, benzothiophenyl, benzimidazolyl, benzoxazolyl, benzothiazolyl, benzotriazolyl, quinolyl, isoquinolyl, purinyl, naphthyridinyl, pteridinyl, and the like. Heteroaryl groups may be unsubstituted or may be substituted with one, two, or three groups selected independently from halogen, OH, C₁-C₆ alkoxy, and C₁-C₆ alkyl.

As used herein, the term “heterocyclyl”, used alone or as part of a larger moiety, e.g., “(heterocyclyl)alkyl”, refers to a univalent stable 4- to 7-membered monocyclic or 7- to 10-membered bicyclic heterocyclic moiety that is either saturated or partially unsaturated, and has, in addition to ring carbon atoms, one to four heteroatoms. Examples of heterocycyl groups include tetrahydrofuranyl, pyrrolidinyl, tetrahydropyranyl, piperidinyl, morpholinyl, and the like. Heterocyclyl groups may be unsubstituted or may be substituted with one, two, or three groups selected independently from halogen, OH, C₁-C₆ alkoxy, and C₁-C₆ alkyl.

Unless otherwise specified, the word “includes” (or any variation thereon, e.g., “include”, “including”, etc.) is intended to be open-ended. For example, “A includes 1, 2 and 3” means that A includes but is not limited to 1, 2 and 3.

As used herein, the term “Not Active” means that the exhibited efficacy is less than 10%.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, methanesulfonate, 2-naphthalenesulfonate, nicotinate, nitrate, oleate, palmitate, pamoate, pectinate, persulfate, 3-phenylpropionate, pivalate, propionate, stearate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “subject” includes a mammal (e.g., a human, in some embodiments including prenatal human forms). In some embodiments, a subject is suffering from a relevant disease, disorder, or condition. In some embodiments, a subject is susceptible to a disease, disorder, or condition. In some embodiments, a subject displays one or more symptoms or characteristics of a disease, disorder, or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is a fetus, an infant, a child, a teenager, an adult, or a senior citizen (i.e., the subject is of advanced age, such as older than 50). In some embodiments, a child refers to a human between two and 18 years of age. In some embodiments, an adult refers to a human eighteen years of age or older.

Unless otherwise specified, the phrase “such as” is intended to be open-ended. For example, “A can be a halogen, such as chlorine or bromine” means that A can be, but is not limited to, chlorine or bromine.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement hydrogen, carbon, nitrogen, oxygen, chlorine, or fluorine with ²H, ³H, ¹¹C, ¹³C, ¹⁴C, ¹³N, ¹⁵N, ¹⁷O, ¹⁸O, ³⁶Cl , or ¹⁸F, respectively, are within the scope of this invention. Such compounds are useful, for example, as analytical tools, as probes in biological assays, or as therapeutic agents in accordance with the present invention. Additionally, incorporation of heavier isotopes such as deuterium (²H) can afford certain therapeutic advantages resulting from greater metabolic stability, for example, increase in vivo half-life, or reduced dosage requirements.

Unless otherwise stated, diastereomeric excess is expressed as % de, i.e., for diastereomers X and Y, the diastereomeric excess of X=((x−y)/(x+y))*100, where x and y are the fractions of X and Y, respectively.

Unless otherwise stated, enantiomeric excess is expressed as % ee, i.e., for enantiomers X and Y, the enantiomeric excess of X=((x−y)/(x+y))*100, where x and y are the fractions of X and Y, respectively.

### Chemical Entities of Formula I

In some embodiments of chemical entities described herein, there are compounds of Formula I or a pharmaceutically acceptable salt thereof

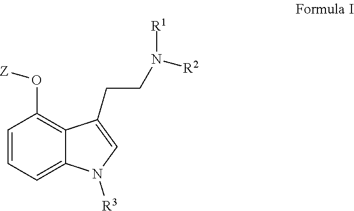

wherein: (i) R¹ is C₁-C₆ alkyl, C₂-C₆ alkenyl, C₂-C₆ alkynyl, C₃-C₆ cycloalkyl, or CH₂(C₃-C₆ cycloalkyl), substituted benzyl, halobenzyl, C₁-C₆ alkyl benzyl, C₁-C₆ alkoxy benzyl or C₁-C₆ alkyl aryl; (ii) R² is H, C₁-C₆ alkyl, C₂-C₆ alkenyl, C₂-C₆ alkynyl, C₃-C₆ cycloalkyl, CH₂(C₃-C₆ cycloalkyl), substituted benzyl, halobenzyl, C₁-C₆ alkyl benzyl, C₁-C₆ alkoxy benzyl or C₁-C₆ alkyl aryl; and (iii) R³ is C₁-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl); and wherein: (A) if R¹ and R² are C₁ alkyl, then R³ is C₂-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl); (B) if R¹ and R³ are C₁ alkyl, then R² is H, C₂-C₆ alkyl, C₂-C₆ alkenyl, C₂-C₆ alkynyl, C₃-C₆ cycloalkyl, CH₂(C₃-C₆ cycloalkyl), or alkylaryl; (C) if R² and R³ are C₁ alkyl, then R¹ is H, C₂-C₆ alkyl, C₂-C₆ alkenyl, C₂-C₆ alkynyl, C₃-C₆ cycloalkyl, or CH₂(C₃-C₆ cycloalkyl); and (D) if R¹ and R² are C₁ alkyl, then R³ is C₁-C₃ alkyl, C₅-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl).

R¹ and R² may be joined to form a 4-7 membered heterocyclyl group.

Z may be H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium such as sodium or potassium.

In some embodiments, the chemical entity of Formula I is a prodrug comprising a prodrug linkage that is known in the art. Such prodrug may comprise at Z an ester linkage (including, but not limited to, carboxyl ester linkage, carbamate ester linkage, carbonate ester linkage, phosphate ester linkage, and amino acid ester linkage). Other suitable prodrugs include hydroxyl prodrugs and phenolic prodrugs.

Chemical entities described herein include those described generally above, and are further illustrated by the classes, subclasses, and species disclosed herein.

In some embodiments: (i) R¹ is C₁-C₄ alkyl; (ii) R² is H, C₂-C₆ alkyl, C₂-C₆ alkenyl, C₂-C₆ alkynyl, C₃-C₆ cycloalkyl, or CH₂(C₃-C₆ cycloalkyl); (iii) R³ is C₁-C₆ alkyl, C₃-C₆ cycloalkyl, CH₂(C₃-C₆ cycloalkyl), 4-7 membered heterocyclyl, or CH₂(4-7 membered heterocyclyl); and (iv) Z is H, (R⁴)(R⁵)N—C(0)-, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium. R¹ and R² may be joined to form a 4-7 membered heterocyclyl group.

In some embodiments: (i) R¹ is C₁-C₄ alkyl; (ii) R² is H, C₂-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group; (iii) R³ is C₁-C₆ alkyl, C₃-C₆ cycloalkyl, CH₂(C₃-C₆ cycloalkyl), 4-7membered heterocyclyl, or CH₂(4-7 membered heterocyclyl); and (iv) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments: R¹ is C₁-C₄ alkyl substituted with 1-3 fluorine atoms; (ii) R² is C₁-C₆ alkyl, C₂-C₆ alkenyl, C₂-C₆ alkynyl, C₃-C₆ cycloalkyl, or CH₂(C₃-C₆ cycloalkyl); (iii) R³ is C₁-C₆ alkyl, C₃-C₆ cycloalkyl, CH₂(C₃-C₆ cycloalkyl), 4-7 membered heterocyclyl, or CH₂(4-7 membered heterocyclyl); and (iv) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷OP(O)—, wherein R⁶ and R⁷ are independently H or a cationic counterion of a phosphate salt form such as sodium or potassium.

In some embodiments: (i) R¹ is C₁-C₄ alkyl; (ii) R² is C₁-C₆ alkyl, C₂-C₆ alkenyl, C₂-C₆ alkynyl, C₃-C₆ cycloalkyl, or CH₂(C₃-C₆ cycloalkyl); (iii) R³ is C₁-C₆ alkyl, C₃-C₆ cycloalkyl, CH₂(C₃-C₆ cycloalkyl), 4-7 membered heterocyclyl, or CH₂(4-7 membered heterocyclyl); and (iv) Z is (R⁴)(R⁵)N—C(O)—, wherein R⁴ and R⁵ are independently chosen from H, C₁-C₄ alkyl, and C₃-C₆ cycloalkyl, and which may be joined to form a 4-7 membered heterocyclyl group.

In some embodiments: (i) R¹ is C₁-C₄ alkyl; (ii) R² is C₁-C₆ alkyl, C₂-C₆ alkenyl, C₂-C₆ alkynyl, C₃-C₆ cycloalkyl, or CH₂(C₃-C₆ cycloalkyl); (iii) R³ is C₁-C₆ alkyl, C₃-C₆ cycloalkyl, CH₂(C₃-C₆ cycloalkyl), 4-7 membered heterocyclyl, or CH₂(4-7 membered heterocyclyl); and (iv) Z is C₁-C₆ alkyl-C(O).

In some embodiments: (i) R¹ is C₁-C₄ alkyl; (ii) R² is C₁-C₆ alkyl, C₂-C₆ alkenyl, C₂-C₆ alkynyl, C₃-C₆ cycloalkyl, or CH₂(C₃-C₆ cycloalkyl); (iii) R³ is C₁-C₆ alkyl, C₃-C₆ cycloalkyl, CH₂(C₃-C₆ cycloalkyl), 4-7 membered heterocyclyl, or CH₂(4-7 membered heterocyclyl); and (iv) Z is C₃-C₆ cycloalkyl-C(O);

In some embodiments: (i) R¹ is C₁-C₄ alkyl; (ii) R² is C₁-C₆ alkyl, C₂-C₆ alkenyl, C₂-C₆ alkynyl, C₃-C₆ cycloalkyl, or methyl CH₂(cycloalkyl); (iii) R³ is C₂-C₆ alkyl, C₃-C₆ cycloalkyl, CH₂(₃-C₆ cycloalkyl), 4-7 membered heterocyclyl, or CH₂(4-7 membered heterocyclyl); and (iv) Z is (R⁶O)(R⁷O)P(O)—, wherein R⁶ and R⁷ are independently H or a cationic counterion of a phosphate salt form such as sodium or potassium.

In some embodiments: (i) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), wherein if R¹ is C₁ alkyl then R² is chosen from H, C₂-C₆ alkyl, C₂-C₆ alkenyl, C₂-C₆ alkynyl, C₃-C₆ cycloalkyl, or CH₂(₃-C₆ cycloalkyl); (ii) R³ is C₁-C₆ alkyl; and (iv) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium. R¹ and R² may be joined to form a 4-7 membered heterocyclyl group

In some embodiments: (i) R³ is ethyl; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group; and (iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium. Compounds comprising an ethyl group at the R³ position exhibited an unexpected improved 5-HT2C receptor selectivity.

In some embodiments: (i) R³ is C₁-C₆ alkyl substituted with 1-3 fluorine atoms; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group; and (iii) Z is H.

In some embodiments: (i) R³ is C₁-C₆ alkyl substituted with 1-3 fluorine atoms; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group; and (iii) Z is (R⁴)(R⁵)N—C(O)—, wherein R⁴ and R⁵ are independently chosen from H, C₁-C₄ alkyl, and C₃-C₆ cycloalkyl, and which may be joined to form a 4-7 membered heterocyclyl group.

In some embodiments: (i) R³ is C₁-C₆ alkyl substituted with 1-3 fluorine atoms; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group, and R¹ and R² cannot both be H; and (iii) Z is C₁-C₆ alkyl-C(O) or C₃-C₆ cycloalkyl-C(O).

In some embodiments: (i) R³ is C₁-C₆ alkyl substituted with 1-3 fluorine atoms; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group; and (iii) Z is (R⁶O)(R⁷O)P(O)—, wherein R⁶ and R⁷ are independently H or a cationic counterion of a phosphate salt form such as sodium or potassium.

In some embodiments: (i) R³ is cyclopropyl; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group, wherein R¹ and R² cannot both be H; and (iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium. Compounds comprising a cyclopropyl group at the R³ position have shown potential for unexpected improved 5-HT2C receptor selectivity.

In some embodiments: (i) R³ is cyclobutyl; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group, wherein R¹ and R² cannot both be H; and (iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments: (i) R³ is 3-oxetanyl; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group, wherein R¹ and R² cannot both be H; and (iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₃-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments: (i) R³ is cyclopropylmethyl; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group, wherein R¹ and R² cannot both be H; and (iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments: (i) R³ is cyclobutylmethyl; (ii) R¹ and R² are independently chosen from H, C₁-C₆ alkyl, C₂-C₆ alkenyl, C₂-C₆ alkynyl, C₃-C₆ cycloalkyl, or CH₂(C₃-C₆ cycloalkyl), and R¹ and R² may be joined to form a 4-7 membered heterocyclyl group, wherein R¹ and R² cannot both be H; and (iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments: (i) R¹ is alkylaryl (ii) R² is H (iii) R³ is C₁-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl) iii) Z is H, (R⁴)(R⁵)N—C(O)—, C₁-C₆ alkyl-C(O), or C₃-C₆ cycloalkyl-C(O), wherein R⁴ and R⁵ are independently chosen from H, C₃-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments (i) R¹ is CH₂-aryl (ii) R² is H (iii) R³ is C₁-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl) iv) Z is H, (R⁴)(R⁵)N—C(O)—, C₁-C₆ alkyl-C(O), or C₃-C₆ cycloalkyl-C(O), wherein R⁴ and R⁵ are independently chosen from H, C₃-C₄ alkyl, and C₃-C₆ cycloalkyl, and which may be joined to form a 4-7 membered heterocyclyl group; or Z is aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

In some embodiments (i) R¹ is CH₂-aryl (ii) R² is H (iii) R³ is C₁-C₄ alkyl iv) Z is H, independently chosen from H, C₃-C₄ alkyl, and C₃-C₆ cycloalkyl, and which may be Z is C₁-C₆ alkyl-C(O), aryl-C(O) or heteroaryl-C(O); or Z is (R⁶O)(R⁷O)P(O)—, wherein R⁶ and R⁷ are independently H or a cationic counterion of a phosphate salt form such as sodium or potassium.

Examples of chemical entities of Formula I are shown in Table 2 below.

TABLE 2
Examples of Compounds of Formula I.

| | 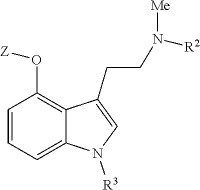 | | |
|---|---|---|---|
| compound | R² | R³ | Z |
| 1 (prior art) | CH₃ | CH₃ | H |
| 2 | CH₃ | CD₃ | H |
| 3 | CH₃ | CH₂CH₃ | H |
| 4 | CH₃ | CH₂CHF₂ | H |
| 5 | CH₃ | CH₂CH₂CH₃ | H |
| 6 (prior art) | CH₃ | CH₂CH₂CH₂CH₃ | H |
| 7 | CH₃ | CH₂CH₂CH═CH₂ | H |
| 8 | CH₃ | CH(CH₃)₂ | H |
| 9 | CH₃ | cyclopropyl | H |
| 10 | CH₃ | cyclopropylmethyl | H |
| 11 | CH₃ | cyclobutyl | H |
| 12 | CH₃ | 3-oxetanyl | H |
| | 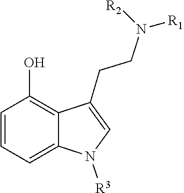 | | |
| compound | R¹ | R² | R³ |
| 200 | CH₃ | CH₂CF₂H | CH₃ |
| 201 | CH₃ | CH₂CF₃ | CH₃ |
| 202 | CH₃ | CH₂CH₃ | CH₃ |
| 203 | CH₃ | CH₂(CH₃)₂ | CH₃ |
| 204 | CH₃ | CH₂(cyclopropyl) | CH₃ |
| 205 | CH₂CH₃ | CH₂CH₃ | CH, |
| 206 | CH₂(CH₃)₂ | CH₂(CH₃)₂ | CH₃ |
| 207 | CH₂CF₂H | H | CH₃ |
| 208 | CH₂CF₃ | H | CH₃ |
| 209 | 2-methoxybenzyl | H | CH₃ |
| 210 | 2-methylbenzyl | H | CH₃ |
| 211 | 2-fluorobenzyl | H | CH₃ |
| 212 | 2-chlorobenzyl | H | CH₃ |
| 213 | 2-methoxybenzyl | H | CH₂CH₃ |
| 214 | 2-methylbenzyl | H | CH₂CH₃ |
| 215 | 2-fluorobenzyl | H | CH₂CH₃ |
| 216 | 2-chlorobenzyl | H | CH₂CH₃ |
| 217 | 3-methoxybenzyl | H | CH₃ |
| 218 | 3-methylbenzyl | H | CH₃ |
| 219 | 3-fluorobenzyl | H | CH₃ |
| 220 | 3-chlorobenzyl | H | CH₃ |
| 221 | 3-methoxybenzyl | H | CH₂CH₃ |
| 222 | 3-methylbenzyl | H | CH₂CH₃ |
| 223 | 3-fluorobenzyl | H | CH₂CH₃ |
| 224 | 3-chlorobenzyl | H | CH₂CH₃ |

Note that with regard to prior art compound 1, the inventors identified in this application found similar functional potency in at human 5-HT2C receptors (ca. 10 nM Table X) as that reported in the prior art, but have also surprisingly found about 86 nM or 10-fold higher potency and higher efficacy (Table 4) at 5-HT2A receptors than had been reported. In addition, the inventors identified in this application have found surprisingly high potency and selectivity for 5-HT2C receptors with 1-ethyl (e.g. compound 3) (Table 4).

### Method of Chemical Synthesis

Chemical entities of formula I can be synthesized according to Scheme 1 and/or using methods known in the art. As depicted in Scheme 1 in a first step 4-(benzyloxy)indole is treated with oxalyl chloride in an inert aprotic organic solvent such as diethyl ether. The resulting intermediate is trapped “in situ” with the requisite amine to yield compounds of Intermediate-1 structure. Intermediate-1 compounds are reduced with LiAIH₄ in an inert solvent system such as a THF-dioxane mixture and heated to give a 4-benzyloxytryptamine compounds of Intermediate-2 structure. The indole nitrogen is then alkylated with a requisite alkyl halide for example an alkyl iodide of formula R³-I after deprotonation with a base such as NaH in a suitable organic solvent such as DMF to give compounds of Intermediate-3 structure. Hydrogenation of compounds of Intermediate-3 structure then gives compounds of the invention of Formula (I) wherein Z═OH. Compounds of formula I in which R³ is cyclopropyl can be prepared in analogous fashion as per Scheme 1 starting from 4-(benzyloxy)-1-cyclopropyl-1H-indole.

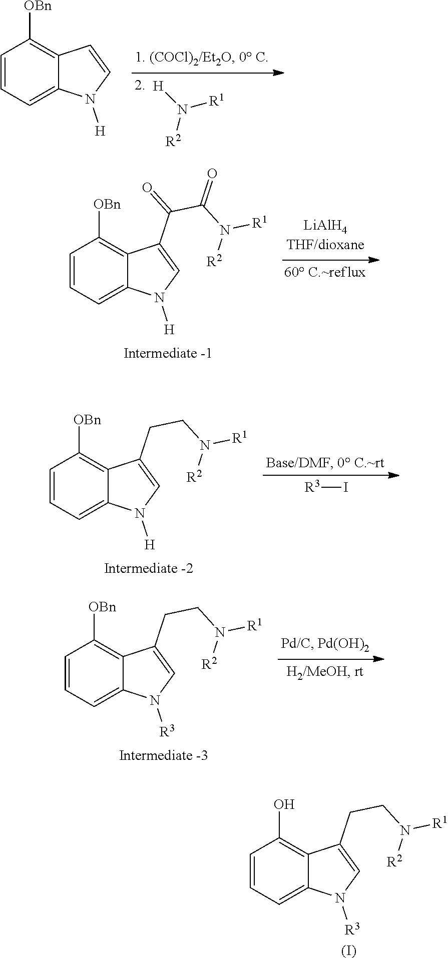

Scheme 1: Prodrugs of compounds of Formula I where Z is a phosphate group can be synthesized from compounds of formula I where Z═H using methods that are known in the art. Scheme 2 is an example of such method for yielding the phosphate in acid form which may be partially or fully neutralized with bases to deliver the corresponding salt forms.

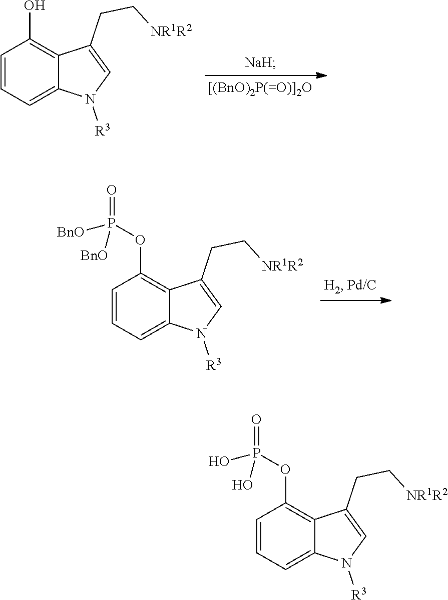

Scheme 2: Prodrugs of compounds of formula I where Z is an acyl group can be synthesized from compounds of formula I where Z═H using methods that are known in the art. Standard procedures include treating compounds of Formula I with an anhydride or acid chloride such as acetic anhydride or pivaloyl chloride in an inert organic solvent such as dichloromethane in the presence of a tertiary amine base such as triethylamine.

Scheme 3: Prodrugs of compounds of formula I where Z comprises a carbamate group can be synthesized from compounds of formula I where Z═H using methods that are known in the art. Standard procedures include treating compounds of Formula I with an isocyanate (Scheme 3) such as isopropyl isocyanate or a carbamoylchloride such as dimethylcarbamyl chloride in an inert organic solvent such as dichloromethane in the presence of a tertiary amine base such as triethylamine.

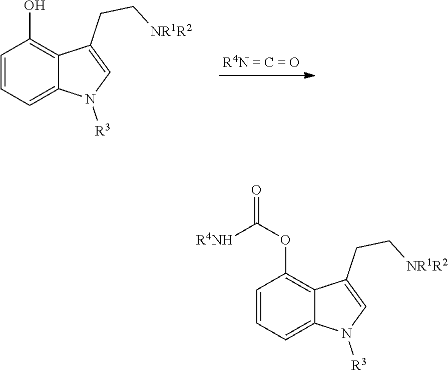

Examples of compounds synthesized herein are provided in Table 3 as follows.

TABLE 3
Examples of Compounds of Formula I Synthesized

| | 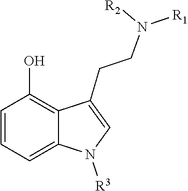 | | |
|---|---|---|---|
| Ex. # | R² | R³ | R¹ |
| 1 (prior art) | CH₃ | CHa | CH₃ |
| 2 | CH₃ | CH₂CH₃ | CH₃ |
| 3 | CH₃ | CH₂CF₂H | CH₃ |
| 4 | CH₃ | CH₂CH₂CH₃ | CH₃ |
| 5 | CH₃ | CH(CH₃)₂ | CH₃ |
| 6 (prior art) | CH₃ | CH₂CH₂CH₂CH₃ | CH₃ |
| 7 | CH₃ | cyclopropyl | CH₃ |
| 8 | CH₃ | CH₂(cyclopropyl) | CH₃ |
| 9 | CH₃ | cyclobutyl | CH₃ |
| 10 | CH₃ | 3-oxetanyl | CH₃ |
| 11 | CH₃ | CH₂CH₂CH═CH₂ | CH₃ |
| 12 | CH₂CH₃ | CH₃ | CH₃ |
| 13 | CH(CH₃)₂ | CH₃ | CH₃ |
| 14 | CH(CH₃)₂ | CH₃ | CH(CH₃)₂ |
| 15 | CH₂CF₃ | CH₃ | CH₃ |
| 16 | CH₂(cyclopropyl) | CH₃ | CH₃ |
| 17 | CH₂CF₂H | CH₃ | CH₃ |
| 18 | CH₂CH₃ | CH₃ | CH₂CH₃ |
| 19 | CH₃ | CD₃ | CH₃ |
| 20 | CH₂CH₂CH₃ | CH₃ | CH₃ |
| 21 | CH₂(cyclopropyl) | CH₃ | H |
| 22 | CH₂CF₂H | CH₃ | H |
| 23 | CH₂CF₃ | CH₃ | H |
| 24 | 2-OMeBn | CH₃ | H |
| 25 | 2-OMeBn | CH₃ | CH₃ |

As depicted in the Examples below, chemical entities are prepared according to the following procedures. It will be appreciated that, although the general methods depict the synthesis of certain chemical entities disclosed herein, the following methods, and other methods known to persons skilled in the art, can be applied to all chemical entities and subclasses and species of each of these chemical entities, as described herein.

Temperatures are given in degrees centigrade. If not mentioned otherwise, all evaporations are performed under reduced pressure, preferably between 15 mm Hg and 100 mm Hg. The structures of intermediates and final products are confirmed by standard analytical methods, for example, mass spectrometry and NMR spectroscopy.

Abbreviations used in the Examples below are summarized in Table 4 as follows:

TABLE 4
Abbreviations.

| Abbreviation | Meaning |
|---|---|
| aq | aqueous |
| aq NH₃ 25% | ammonia solution in water |
| Ac | acetyl |
| Boc | t-butoxycarbonyl |
| Cbz | benzyloxycarbonyl |
| DCM | dichloromethane |
| DCE | 1,2-dichloroethane |
| DIPEA | N,N-diisopropylethylamine |
| DMAP | 4-(dimethylamino)pyridine |
| DMF | N,N-dimethylformamide |
| DMSO | dimethyl sulfoxide |
| Et | ethyl |
| EtO | diethyl ether (“ether”) |
| EtOAc | ethyl acetate |
| EtOH | ethanol |
| Eq | equivalent(s) |
| G | gram(s) |
| H | hour(s) |
| HCI | hydrochloric acid |
| HPLC | high performance liquid chromatography |
| i-Pr | isopropyl |
| LC | liquid chromatography |
| mg | milligrams |
| Me | methyl |
| MHz | megahertz |
| Mel | methyliodide |
| mL | milliliters |
| MeOH | methanol |
| MS | mass spectrometry |
| MS (ESI) | mass spectrometry in electrospray ionization mode |
| MTBE | methyl tert-butyl ether |
| NaHMDS | sodium hexamethyldisilazide |
| NMP | N-methyl-2-pyrrolidone |
| NMR | nuclear magnetic resonance |
| PE | petroleum ether |
| rt | room temperature |
| t-Bu | tert-butyl |
| TEA | triethylamine |
| TFA | trifluoroacetic acid |
| THF | tetra hydrofuran |
| TLC | thin layer chromatography |
| Ts | p-toluenesulfonyl |

Synthesis of Intermediate A: 2-(4-(benzyloxy)-1H-indol-3-yl)-N,N-dimethylethan-1-amine

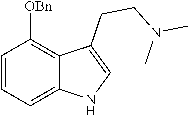 [SMILES: CN(C)CCc1c[nH]c2cccc(OCc3ccccc3)c12]

Step 1: 2-(4-(benzyloxy)-1H-indol-3-yl)-N,N-dimethyl-2-oxoacetamide

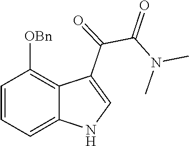 [SMILES: CN(C)C(=O)C(=O)c1c[nH]c2cccc(OCc3ccccc3)c12]

To a solution of 4-(benzyloxy)-1H-indole (5.0 g, 22.4 mmol) in ether (100 mL) under ice-salt water bath cooling was added dropwise a solution of oxalyl chloride (5.80 g, 45.7 mmol) in ether (45 mL) under nitrogen atmosphere. The resulting brown suspension was stirred for 3 h, then added dropwise into 40% aq. dimethylamine (50 mL) under ice-salt water bath cooling. The resulting suspension was stirred overnight at room temperature. The suspension was filtered, and the filter cake was rinsed with water. The filter cake was dried under reduced pressure, and then slurried in hexane (50 mL). After stirrring at room temperature for 1 h, the slurry was filtered to afford the title product as an orange solid (3.81 g, 52%).

Step 2: 2-(4-(benzyloxy)-1H-indol-3-yl)-N,N-dimethylethan-1-amine

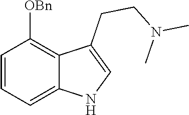 [SMILES: CN(C)CCc1c[nH]c2cccc(OCc3ccccc3)c12]

To a slurry of LiAlH₄ (2.43 g, 63.9 mmol) in anhydrous THF (27 mL) under nitrogen atmosphere was added dioxane (54 mL) at room temperature, and the resulting suspension was heated to 60° C. on an oil bath. A solution of 2-(4-(benzyloxy)-1H-indol-3-yl)-N,N-dimethyl-2-oxoacetamide (3.81 g, 11.8 mmol) in THF/dioxane (5/3, 105 mL) was then added dropwise. The resulting mixture was heated to 70° C. and stirred for 4 h. The reaction mixture was further heated to 110° C. and stirred overnight. After cooling to room temperature, a solution of water (7 mL) in THF (30 mL) was added dropwise into the mixture to give a gray suspension. Ether (25 mL) was added and the resulting mixture was stirred at room temperature for 1h then filtered. The filter cake was rinsed several times with THF. The filtrates were combined and concentrated under reduced pressure. The resulting residue was purified by silica gel column chromatography (DCM/MeOH/aq. NH₃=30/1/0.5) to afford the title product as an off-white solid (2.85 g, 82%). ¹H NMR (400 MHz, CDC1₃) δ 8.10 (br, 1H), 7.50 (d, J=7.2 Hz, 2H), 7.40-7.35 (m, 2H), 7.34-7.29 (m, 1H), 7.05 (t, J=8.0 Hz, 1H), 6.95 (d, J=7.6 Hz, 1H), 6.88 (d, J=2.4 Hz, 1H), 6.54 (d, J=7.6 Hz, 1H), 5.19 (s, 2H), 3.07-3.03 (m, 2H), 2.61-2.57 (m, 2H), 2.14 (s, 6H).

Example 1: 3-(2-(dimethylamino)ethyl)-1-methyl-1H-indol-4-ol was prepared from 2-(4-(benzyloxy)-1H-indol-3-yl)-N,N-dimethylethan-1-amine by the analogous general procedure used to prepare 1-butyl-3-(2-(dimethylamino)ethyl)-1H-indol-4-ol (see Example 6).

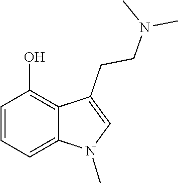 [SMILES: CN(C)CCc1cn(C)c2cccc(O)c12]

Step 1: 2-(4-(benzyloxy)-1-methyl-1H-indol -3-yl)-N,N-dimethylethan-1-amine

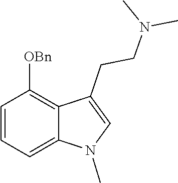 [SMILES: CN(C)CCc1cn(C)c2cccc(OCc3ccccc3)c12]

Following the analogous procedure used in Example 6 starting from the intermediate 2-(4-(benzyloxy)-1H-indol-3-yl)-N,N-dimethylethan-1-amine and using methyl iodide as the the alkyating agent the title crude product was obtained as a light brown oil (466 mg, 29%) and used directly in the next step.

Step 2: 3-(2-(dimethylamino)ethyl)-1-methyl-1H-indol-4-ol

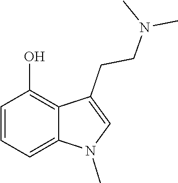 [SMILES: CN(C)CCc1cn(C)c2cccc(O)c12]

The 2-(4-(benzyloxy)-1-methyl-1H-indol-3-yl)-N,N-dimethylethan-1-amine from the previous step was directly hydrogenated following the analogous procedure to Example 6.

The crude product was purified by silica gel column chromatography (eluent: DCM/MeOH/aq. NH₃=50/1/0.5) to afford the title product as an off-white solid (222 mg, 67%). MS (ESI) calcd for C₁₃H₁₈N₂O 218.1; found: 219.3 [M+1]. ¹H NMR (400 MHz, CDCl₃) δ 7.09 (t, J=8.0 Hz, 1 H), 6.78 (d, J=8.0 Hz, 1H), 6.68 (s, 1H), 6.55 (d, J=7.6 Hz, 1H), 3.67 (s, 3H), 2.94-2.90 (m, 2H), 2.70-2.67 (m, 2H), 2.38 (s, 6H).

Example 2: 3-(2-(dimethylamino)ethyl)-1-ethyl-1H-indol-4-ol

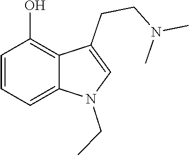 [SMILES: CCn1cc(CCN(C)C)c2c(O)cccc21]

Step 1: 2-(4-(benzyloxy)-1-ethyl-1H-indol-3-yl)-N,N-dimethylethan-1-amine

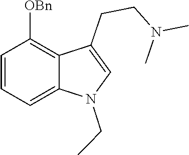 [SMILES: CCn1cc(CCN(C)C)c2c(OCc3ccccc3)cccc21]

Following the analogous procedure used in Example 6 starting from the intermediate 2-(4-(benzyloxy)-1H-indol-3-yl)-N,N-dimethylethan-1-amine and using ethyl iodide as the the alkylating agent the title crude product was obtained as a light brown oil (347 mg, 52%) and used directly in the next step. ¹H NMR (400 MHz, CDCl₃) δ 7.51-7.49 (m, 2H), 7.40-7.36 (m, 2H), 7.33-7.30 (m, 1H), 7.07 (t, J=8.0 Hz, 1H), 6.92 (d, J=8.4 Hz, 1H), 6.82 (s, 1H), 6.53 (d, J=8.0 Hz, 1H), 5.18 (s, 2H), 4.07 (q, J=7.2 Hz, 2H), 3.07-3.03 (m, 2H), 2.63-2.59 (m, 2H), 2.14 (s, 6H), 1.42 (t, J=7.2 Hz, 3H).

Step 2: 3-(2-(dimethylamino)ethyl)-1-ethyl-1H-indol-4-ol

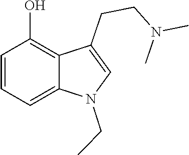 [SMILES: CCn1cc(CCN(C)C)c2c(O)cccc21]

The 2-(4-(benzyloxy)-1-ethyl-1H-indol-3-yl)-N,N-dimethylethan-1-amine from the previous step was hydrogenated following the analogous procedure to Example 6. The crude product was purified by silica gel column chromatography (eluent: DCM/MeOH/aq. NH₃=50/1/0.5) to afford the title product as a light brown solid (200 mg, 80%). MS (ESI) calcd for C₁₄H₂₀N₂O: 232.2; found: 233.5 [M+1]. ¹H NMR (400 MHz, CDCl₃) δ 7.07 (t, J=8.0 Hz, 1H), 6.81 (d, J=7.6 Hz, 1H), 6.74 (s, 1H), 6.54 (d, J=7.6 Hz, 1H), 4.05 (q, J=7.2 Hz, 2H), 2.94-2.91 (m, 2H), 2.70-2.68 (m, 2H), 2.38 (s, 6H), 1.42 (t, J=7.2 Hz, 3H).

Example 3: 1-(2,2-difluoroethyl)-3-(2-(dimethylamino)ethyl)-1H-indol-4-ol

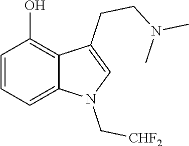 [SMILES: CN(C)CCc1cn(CC(F)F)c2cccc(O)c12]

Step 1: 2-(4-(benzyloxy)-1-(2,2-difluoroethyl)-1H-indol-3-yl)-N,N-dimethylethan-1-amine

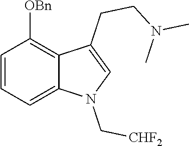 [SMILES: CN(C)CCc1cn(CC(F)F)c2cccc(OCc3ccccc3)c12]

Following the analogous procedure, used in Example 6 starting from the intermediate 2-(4-(benzyloxy)-1H-indol-3-yl)-N,N-dimethylethan-1-amine and using 2-bromo-1,1-difluoroethane as the the alkyating agent the title crude product was obtained as a light brown oil (240 mg, 49%) and used directly in the next step. ¹H NMR (400 MHz, CDCI₃) δ 7.51-7.47 (m, 2H), 7.41-7.35 (m, 2H), 7.35-7.30 (m, 1H), 7.12 (t, J=8.0 Hz, 1H), 6.91 (d, J=8.4 Hz, 1H), 6.81 (s, 1H), 6.58 (d, J=8.0 Hz, 1H), 5.95 (tt, J=55.6 and 4.0 Hz, 1H), 5.19 (s, 2H), 4.35 (dt, J=4.0 and 14.0 Hz, 2H), 3.06-3.00 (m, 2H), 2.60-2.56 (m, 2H), 2.14 (s, 6H).

Step 2: 1-(2,2-difluoroethyl)-3-(2-(dimethylamino)ethyl)-1H-indol-4-ol

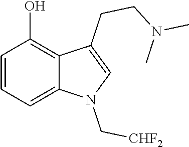 [SMILES: CN(C)CCc1cn(CC(F)F)c2cccc(O)c12]

The 2-(4-(benzyloxy)-1-(2,2-difluoroethyl)-1H-indol-3-yl)-N,N-dimethylethan-1-amine from the previous step was hydrogenated following the analogous procedure to Example 6. The crude product was purified by silica gel column chromatography (eluent: DCM/MeOH/aq. NH₃.H₂O=50/1/0.5) to afford the title product as an off-white solid (103 mg, 56%). MS (ESI) calcd for C₁₄H₁₈F₂N₂O: 268.1; found: 269.4 [M+1].¹H NMR (400 MHz, CDCl₃) δ 7.11 (t, J=7.8 Hz, 1H), 6.79 (d, J=8.0 Hz, 1H), 6.75 (s, 1H), 6.59 (d, J=7.2 Hz, 1H), 5.97 (tt, J=4.0 and 55.6 Hz, 1H), 4.32 (dt, J=4.4 and 14.0 Hz, 2H), 2.94-2.90 (m, 2H), 2.72-2.68 (m, 2H), 2.38 (s, 6H).

Example 4: 3-(2-(dimethylamino)ethyl)-1-propyl-1H-indol-4-ol

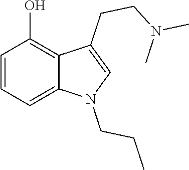 [SMILES: CCCn1cc(CCN(C)C)c2c(O)cccc21]

Step 1: 2-(4-(benzyloxy)-1-propyl-1H-indol-3-yl)-N,N-dimethylethan-1-amine

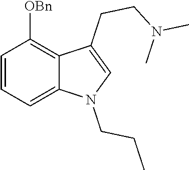 [SMILES: CCCn1cc(CCN(C)C)c2c(OCc3ccccc3)cccc21]

Following the analogous procedure used in Example 6 starting from the intermediate 2-(4-(benzyloxy)-1H-indol-3-yl)-N,N-dimethylethan-1-amine and using 1-iodopropane as the the alkyating agent the title crude product was obtained as a light yellow oil (150 mg, 51%) and used directly in the next step. ¹H NMR (400 MHz, CDCl₃) δ 7.52-7.48 (m, 2H), 7.42-7.35 (m, 2H), 7.35-7.29 (m, 1H), 7.06 (t, J=8.0 Hz, 1H), 6.92 (d, J=8.4 Hz, 1H), 6.80 (s, 1H), 6.53 (d, J=7.6 Hz, 1H), 5.17 (s, 2H), 3.98 (t, J=7.0 Hz, 2H), 3.07-3.03 (m, 2H), 2.64-2.60 (m, 2H), 2.14 (s, 6H), 1.87-1.78 (m, 2H), 0.92 (t, J=7.4 Hz, 3H).

Step 2: 3-(2-(dimethylamino)ethyl)-1-propyl-1H-indol-4-ol

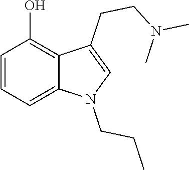 [SMILES: CCCn1cc(CCN(C)C)c2c(O)cccc21]

The 2-(4-(benzyloxy)-1-propyl-1H-indol-3-yl)-N,N-dimethylethan-1-amine from the previous step was hydrogenated following the analogous procedure to Example 6. The crude product was purified by silica gel column chromatography (eluent: DCM/MeOH/aq. NH₃.H₂O=40/1/0.5) to afford the title product as a light brown solid (59 mg, 29%). MS (ESI) calcd for C₁₅H₂₂N₂O 246.2; found: 247.5 [M+1]. ¹H NMR (400 MHz, CDCl₃) δ 7.06 (t, J=8.0 Hz, 1H), 6.80 (d, J=8.0 Hz, 1H), 6.73 (s, 1H), 6.54 (t, J=7.6 Hz, 1H), 3.96 (t, J=7.2 Hz, 2H), 2.93-2.91 (m, 2H), 2.70-2.67 (m, 2H), 2.38 (s, 6H), 1.88-1.78 (m, 2H), 0.93 (t, J=7.2 Hz, 3H).

Example 5: 3-(2-(dimethylamino)ethyl)-1-isopropyl-1H-indol-4-ol

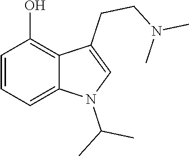 [SMILES: CC(C)n1cc(CCN(C)C)c2c(O)cccc21]

Step 1: 2-(4-(benzyloxy)-1-isopropyl-1H-indol-3-yl)-N,N-dimethylethan-1-amine

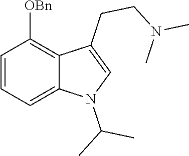 [SMILES: CC(C)n1cc(CCN(C)C)c2c(OCc3ccccc3)cccc21]

Following the analogous procedure used in Example 6 starting from the intermediate 2-(4-(benzyloxy)-1H-indo1-3-yl)-N,N-dimethylethan-1-amine and using 2-bromopropane as the alkylating agent. The crude product was purified by silica gel column chromatography (eluent: DCM/MeOH/aq. NH₃=40/1/0.5) to afford the title product as a light yellow oil (284 mg, 62%). ¹H NMR (400 MHz, CDCl₃) δ 7.53-7.48 (m, 2H), 7.43-7.33 (m, 3H), 7.10 (t, J=8.0 Hz, 1H), 7.00-6.95 (m, 2H), 6.57 (d, J=8.0 Hz, 1H), 5.14 (s, 2H), 4.63-4.53 (m, 1H), 3.16-3.11 (m, 2H), 2.84-2.79 (m, 2H), 2.19 (s, 6H), 1.48 (d, J=6.8 Hz, 6H).

Step 2: 3-(2-(dimethylamino)ethyl)-1-isopropyl-1H-indol-4-ol (BMB-A14)

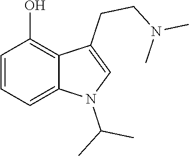 [SMILES: CC(C)n1cc(CCN(C)C)c2c(O)cccc21]

The 2-(4-(benzyloxy)-1-isopropyl-1H-i ndol1-3-yl)-N , N-dimethylethan-1-amine from the previous step was hydrogenated following the analogous procedure to Example 6. The crude product was purified by silica gel column chromatography (eluent: DCM/MeOH/aq. NH₃ =40/1/0.5) to afford the title product as a light brown solid (104 mg, 49%). MS (ESI) calcd for C₁₅H₂₂N₂O 246.2; found: 247.5 [M+1]. ¹H NMR (400 MHz, CDCl₃) δ 7.06 (t, J=8.0 Hz, 1H), 6.85-6.82 (m, 2H), 6.54 (d, J=7.6 Hz, 1H), 4.59-4.53 (m, 1H), 2.95-2.92 (m, 2H), 2.70-2.67 (m, 2H), 2.38 (s, 6H), 1.47 (t, J=6.8 Hz, 6H).

Example 6: 1-butyl-3-(2-(dimethylamino)ethyl)-1H-indol-4-ol

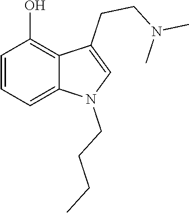 [SMILES: CCCCn1cc(CCN(C)C)c2c(O)cccc21]

Step 1: 2-(4-(benzyloxy)-1-butyl-1H-indol1-3-yl)-N,N-dimethylethan-1-amine

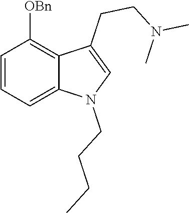 [SMILES: CCCCn1cc(CCN(C)C)c2c(OCc3ccccc3)cccc21]

To a stirred solution of 2-(4-(benzyloxy)-1H-indol-3-yl)-N,N-dimethylethan-1-amine (500 mg, 1.74 mmol) in DMF (4 mL) under ice-water bath cooling was added NaH (60%, 140 mg, 3.48 mmol) in one portion. The resulting mixture was stirred for 30 min then 1-iodobutane (360 mg, 1.74 mmol) was added. The resulting mixture was stirred at room temperature for 2 h then quenched with ice-water and partitioned between ethyl acetate and water. The organic phase was separated, washed with brine, dried over Na₂SO₄, and concentrated under reduced pressure. The residue was purified by silica gel column chromatography (eluent: DCM/MeOH/aq. NH₃=40/1/0.5) to afford the title product as a colorless oil (268 mg, 45%). ¹H NMR (400 MHz, CDCl₃) δ 7.50 (d, J=7.2Hz, 2H), 7.38 (t, J=6.8 Hz, 2H), 7.33-7.29 (m, 1H), 7.06 (t, J=8.0 Hz, 1H), 6.91 (d, J=8.0 Hz, 1H), 6.79 (s, 1H), 6.52 (d, J=7.6 Hz, 1H), 5.18 (s, 2H), 4.01 (t, J=7.2 Hz, 2H), 3.06-3.02 (m, 2H), 2.62-2.58 (m, 2H), 2.14 (s, 6H), 1.81-1.74 (m, 2H), 1.38-1.28 (m, 2H), 0.93 (t, J=7.2 Hz, 3H).

Step 2: 1-butyl-3-(2-(dimethylamino)ethyl)-1H-indol-4-ol

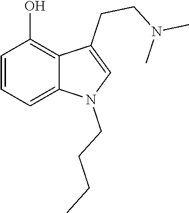 [SMILES: CCCCn1cc(CCN(C)C)c2c(O)cccc21]

A mixture of 2-(4-(benzyloxy)-1-butyl-1H-indol1-3-yl)- N, N-dimethylethan-1-amine (152 mg, 0.43 mmol), 10% Pd/C (30 mg) and 10% Pd(OH)₂/C (30 mg) in methanol (15 mL) was degassed three times with hydrogen. The resulting mixture was then stirred at room temperature overnight under hydrogen atmosphere. The mixture was filtered and concentrated under reduced pressure. The residue was purified by silica gel column chromatography (eluent: DCM/MeOH/aq. NH₃=40/1/0.5) to afford the title product as a light brown solid (63 mg, 56%). MS (ESI) calcd for C₁₆H₂₄N₂O: 260.2; found: 261.5 [M+1].¹H NMR (400 MHz, CDCl₃) δ 7.06 (t, J=8.0 Hz, 1H), 6.80 (d, J=8.0 Hz, 1H), 6.72 (s, 1H), 6.53 (d, J=7.2 Hz, 1H), 3.99 (t, J=7.2 Hz, 2H), 2.94-2.90 (m, 2H), 2.71-2.67 (m, 2H), 2.38 (s, 6H), 1.82-1.74 (m, 2H), 1.37-1.30 (m, 2H), 0.93 (t, J=7.2 Hz, 3H).

Example 7: 1-cyclopropyl-3-(2-(dimethylamino)ethyl)-1H-indol-4-ol hydrochloride

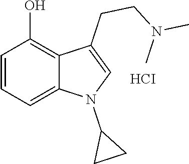

Step 1: 4-(benzyloxy)-1-cyclopropyl-1H-indole

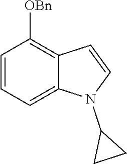 [SMILES: c1ccc(COc2cccc3c2ccn3C2CC2)cc1]

To a stirred suspension of 4-(benzyloxy)-1H-indole (3.01 g, 13.4 mmol), cyclopropylboronic acid (3.52 g, 40.3 mmol), Cu(OAc)₂ (2.50 g, 13.4 mmol) and DMAP (5.0 g, 40 mmol) in anhydrous toluene (72 mL) under nitrogen atmosphere at room temperature was added NaHMDS (7.4 mL, 2.0M, 14.8 mmol). The mixture was heated to 95° C. under oxygen atmosphere, and stirred for 3d until the starting material was consumed. The mixture was filtered through celite, and the filtrate was concentrated under reduced pressure. The residue was triturated several times with petroleum ether. The combined petroleum ether extracts were concentrated, and the residue purified by silca gel column chromatography (eluent: PE/EtOAc=10/1) to afford the title product as a brown oil (1.40 g, 39%). ¹H NMR (400 MHz, CDCl₃) δ 7.51-7.47 (m, 2H), 7.41-7.35 (m, 2H), 7.34-7.28 (m, 1H), 7.20 (d, J=8.4 Hz, 1H), 7.11 (t, J=8.0 Hz, 1H), 7.03 (d, J=3.2 Hz, 1H), 6.61-6.56 (m, 2H), 5.22 (s, 2H), 3.36-3.29 (m, 1H), 1.06-0.99 (m, 4H).

Step 2: 2-(4-(benzyloxy)-1-cyclopropyl-1H-indol1-3-yl)-N,N-dimethyl-2-oxoacetamide

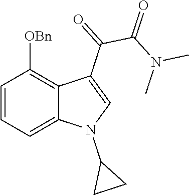 [SMILES: CN(C)C(=O)C(=O)c1cn(C2CC2)c2cccc(OCc3ccccc3)c12]

To a stirred solution of 4-(benzyloxy)-1-cyclopropyl-1H-indole (1.4 g, 5.3 mmol) in ether (40 mL) under ice-salt water bath cooling was added a solution of oxalyl chloride (1.35 g, 10.6 mmol) in ether (10 mL) under nitrogen atmosphere. The resulting brown mixture was stirred for 3 h, then added dropwise into 40% aq. dimethyl amine (10 mL) under ice-salt water bath cooling. The resulting suspension was stirred overnight at room temperature. After the ether was evaporated, the residue was partitioned between DCM and aq. NaHCO₃. The organic phase was washed with brine, dried over Na₂SO₄, and concentrated under reduced pressure. The concentrate was purified by silica gel column chromatography (eluent: PE/EtOAc=2/1) to afford the title product as a light brown solid (1.63 g, 84%).

Step 3: 1-(4-(benzyloxy)-1-cyclopropyl-1H-indol1-3-yl)-2-(dimethylamino)ethan-1-ol

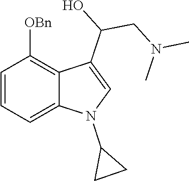 [SMILES: CN(C)CC(O)c1cn(C2CC2)c2cccc(OCc3ccccc3)c12]

To a stirred slurry of LiAlH₄ (874 mg, 23.0 mmol) in anhydrous THF/dioxane (1/2, 48 mL) at 60° C. under nitrogen atmosphere was added a solution of 2-(4-(benzyloxy)-1-cyclopropyl-1H-indol-3-yl)-N,N-dimethyl-2-oxoacetamide (1.6 g, 4.4 mmol) in THF/dioxane (1/2, 60 mL) dropwise. The resulting mixture was heated to 70° C. and stirred for 4 h. The reaction mixture was further heated to 110° C. and stirred overnight. After cooling to room temperature, aqueous THF was added dropwise into the mixture, the mixture was diluted with ether and then filtered. The filter cake was rinsed several times with THF and the combined filtrates were concentrated under reduced pressure. The residue was purified by silica gel column chromatography (eluent: DCM/MeOH/aq. NH3, =97/2/1) to afford the title product as a yellow oil (626 mg, 40%). ¹H NMR (400 MHz, DMSO-d₆) δ 7.54-7.50 (m, 2H), 7.42-7.36 (m, 2H), 7.35-7.30 (m, 1H), 7.12 (d, J=8.0 Hz, 1H), 7.08 (s, 1H), 7.04 (t, J=8.0 Hz, 1H), 6.63 (d, J=7.6 Hz, 1H), 5.20-5.13 (m, 3H), 4.38 (br, 1H), 3.38-3.35 (m, 1H), 2.49-2.45 (m, 1H), 2.35 (dd, J=9.2, 12.4 Hz, 1H), 2.04 (s, 6H), 1.05-1.00 (m, 2H), 0.90-0.85 (m, 2H).

Step 4: 2-(4-(benzyloxy)-1-cyclopropyl-1H-indol-3-yl)-N,N-dimethylethan-1-amine

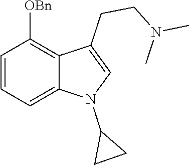 [SMILES: CN(C)CCc1cn(C2CC2)c2cccc(OCc3ccccc3)c12]

To a solution of 1-(4-(benzyloxy)-1-cyclopropyl-1H-indol-3-yl)-2-(dimethylamino)ethan-1-ol (626 mg, 1.79 mmol) in DCM under ice-water bath cooling was added Et₃SiH (2.1 g, 18 mmol). After stirring for 10 min, TFA (1.1 mL, 14 mmol) was added. After stirring for another 2 h, the reaction was quenched with aq. Na₂CO₃. The organic phase was washed with brine, dried over Na₂SO₄, and concentrated under reduced pressure. The concentrate was purified by silica gel column chromatography (eluent: MTBE/MeOH/aq. NH3=97/2/1) to afford the title product as a yellow oil (213 mg, 35%). ¹H NMR (400 MHz, CDCl₃) δ 7.51-7.46 (m, 2H), 7.40-7.34 (m, 2H), 7.33-7.28 (m, 1H), 7.14 (d, J=8.0 Hz, 1H), 7.08 (t, J=8.0 Hz, 1H), 6.81 (s, 1H), 6.54 (d, J=7.6 Hz, 1H), 5.18 (s, 2H), 3.28-3.22 (m, 1H), 3.03-2.97 (m, 2H), 2.59-2.54 (m, 2H), 2.13 (s, 6H), 1.04-0.95 (m, 4H).

Step 5: 1-cyclopropyl-3-(2-(dimethylamino)ethyl)-1H-indol-4-ol hydrochloride

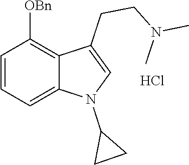 [SMILES: CN(C)CCc1cn(C2CC2)c2cccc(OCc3ccccc3)c12 ; Cl]

A stirred suspension of 2-(4-(benzyloxy)-1-cyclopropyl-1H-indol-3-yl)-N,N-dimethylethan-1-amine (124 mg, 0.37 mmol), 10% Pd/C (30 mg) and 10% Pd(OH)₂/C (30 mg) in methanol (10 mL) was degassed three times with hydrogen. The mixture was stirred at room temperature for 2 h under balloon pressure hydrogen atmosphere. After the starting material was consumed, the mixture was filtered, and the filtrate was concentrated under reduced pressure. The concentrate was purified by silica gel column chromatography (eluent: DCM)/MeOH/aq. NH3=97/2/1) to afford a light yellow oil (55 mg, 62%). The oil was dissolved in DCM (2.0 mL) at room temperature and1M HCl/EtOH (0.35 mL, 0.35 mmol) was added. The resulting solution was stirred for 30 min, and then concentrated under reduced pressure. The residue was triturated with ether to afford the title product as a light brown solid (50 mg, 78%). MS (ESI) calcd for C₁₅H₂₀N₂O: 244.2; found: 245.4 [M+1].¹H NMR (400 MHz, CD₃OD) δ 6.95-6.91 (m, 2H), 6.87 (t, J=8.0 Hz, 1H), 6.32 (d, J=7.2 Hz, 1H), 3.39 (t, J=8.0 Hz, 2H), 3.19-3.12 (m, 3H), 2.81 (s, 6H), 0.98-0.93 (m, 2H), 0.85-0.81 (m, 2H).

Example 8: 1-(cyclopropylmethyl)-3-(2-(dimethylamino)ethyl)-1H-indol-4-ol

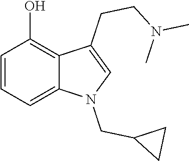 [SMILES: CN(C)CCc1cn(CC2CC2)c2cccc(O)c12]

Step 1: 2-(4-(benzyloxy)-1-(cyclopropylmethyl)-1H-indol-3-yl)-N,N-dimethylethan-1-amine

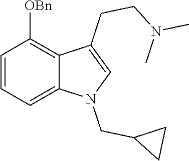 [SMILES: CN(C)CCc1cn(CC2CC2)c2cccc(OCc3ccccc3)c12]

Following the analogous procedure used in Example 6 starting from the intermediate 2-(4-(benzyloxy)-1H-indol-3-yl)-N,N-dimethylethan-1-amine and using (bromomethyl)cyclopropane as the alkylating agent, the title crude product was obtained as a light brown oil (324 mg, 68%) which was used directly in the next step. ¹H NMR (400 MHz, CDCl₃) δ 7.52-7.48 (m, 2H), 7.41-7.36 (m, 2H), 7.35-7.30 (m, 1H), 7.08 (t, J=8.0 Hz, 1H), 6.94 (d, J=8.0 Hz, 1H), 6.92 (s, 1H), 6.54 (d, J=7.6 Hz, 1H), 5.17 (s, 2H), 3.88 (d, J=6.8 Hz, 2H), 3.10-3.04 (m, 2H), 2.69-2.63 (m, 2H), 2.16 (s, 6H), 1.24-1.19 (m, 1H), 0.63-0.57 (m,2H), 0.36-0.32 (m, 2H).

Step 2: 1-(cyclopropylmethyl)-3-(2-(dimethylamino)ethyl)-1H-indol-4-ol

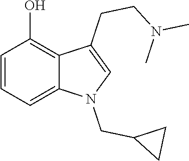 [SMILES: CN(C)CCc1cn(CC2CC2)c2cccc(O)c12]

The 2-(4-(benzyloxy)-1-(cyclopropylmethyl)-1H-indol -3-yl)-N,N-dimethylethan amine from the previous step was hydrogenated following the analogous procedure to Example 6. The crude product was purified by silica gel column chromatography (eluent: DCM/MeOH/aq. NH₃=40/1/0.5) to afford the title product as an off-white solid (64 mg, 26%). MS (ESI) calcd for C₁₆H₂₂N₂O: 258.2; found: 259.5 [M+1].¹H NMR (400 MHz, CDCl₃) δ 7.07 (t, J=8.0 Hz, 1H), 6.85 (s, 1H), 6.82 (d, J=8.0 Hz, 1H), 6.54 (d, J=7.6 Hz, 1H), 2.85 (d, J=6.8 Hz, 2H), 2.96-2.91 (m, 2H), 2.72-2.68 (m, 2H), 2.38 (s, 6H), 1.28-1.23 (m, 1H), 0.63-0.58 (m, 2H), 0.37-0.32 (m, 2H).

Example 9: 1-cyclobutyl-3-(2-(dimethylamino)ethyl)-1H-indol-4-ol

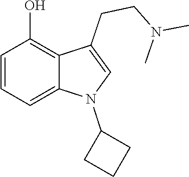 [SMILES: CN(C)CCc1cn(C2CCC2)c2cccc(O)c12]

Step 1: 2-(4-(benzyloxy)-1-cyclobutyl-1H-indol-3-yl)-N,N-dimethylethan-1-amine

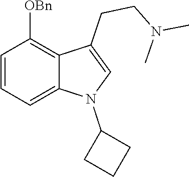 [SMILES: CN(C)CCc1cn(C2CCC2)c2cccc(OCc3ccccc3)c12]

Following the analogous procedure, used in Example 6 starting from the intermediate 2-(4-(benzyloxy)-1H-indol-3-yl)-N,N-dimethylethan-1-amine and using bromocyclobutane as the alkylating agent the title product was obtained as a light brown oil (243 mg, 68%) and used directly in the next step. MS (ESI) calcd for C₂₃H₂₈N₂O 348.2; found: 349.6 [M+1]. ¹H NMR (400 MHz, CDCl₃) δ 7.52-7.48 (m, 2H), 7.45-7.37 (m, 3H), 7.11 (t, J=8.0 Hz, 1H), 7.07 (s, 1H), 6.97 (d, J=8.4 Hz, 1H), 6.60 (d, J=7.2 Hz, 1H), 5.12 (s, 2H), 4.81-4.71 (m, 1H), 3.22-3.16 (m, 2H), 2.98-2.93 (m, 2H), 2.57-2.51 (m, 2H), 2.45-2.35 (m, 2H), 2.23 (s, 6H), 1.94-1.88 (m, 2H).

Step 2: 1-cyclobutyl-3-(2-(dimethylamino)ethyl)-1H-indol-4-ol

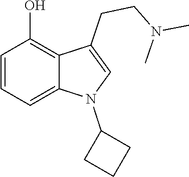 [SMILES: CN(C)CCc1cn(C2CCC2)c2cccc(O)c12]

The 2-(4-(benzyloxy)-1-cyclobutyl-1H-indol-3-yl)-N,N-dimethylethan-1-amine from the previous step was hydrogenated following the analogous procedure to Example 6. The crude product was purified by silica gel column chromatography (eluent: DCM/MeOH/aq. NH₃=40/1/0.5) to afford the title product as a pale purple solid (21 mg, 10%). MS (ESI) calcd for C₁₆H₂₂N₂O 258.2; found: 259.5 [M+1]. ¹H NMR (400 MHz, CDCl₃) δ 7.05 (t, J=8.0 Hz, 1H), 6.89 (s, 1H), 6.81 (d, J=8.4 Hz, 1H), 6.54 (d, J=7.6 Hz, 1H), 4.79-4.71 (m, 1H), 2.96-2.91 (m, 2H), 2.71-2.66 (m, 2H), 2.56-2.49 (m, 2H), 2.45-2.35 (m, 8H), 1.93-1.85 (m, 2H).

Example 10: 3-(2-(dimethylamino)ethyl)-1-(oxetan-3-yl)-1H-indol-4-ol

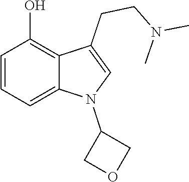 [SMILES: CN(C)CCc1cn(C2COC2)c2cccc(O)c12]

Step 1: 2-(4-(benzyloxy)-1-(oxetan-3-yl)-1H-indol -3-yl)-N,N-dimethylethan-1-amine

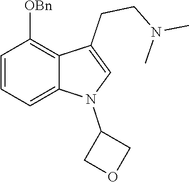 [SMILES: CN(C)CCc1cn(C2COC2)c2cccc(OCc3ccccc3)c12]

Following the analogous procedure used in Example 6 starting from the intermediate 2-(4-(benzyloxy)-1H-indol-3-yl)-N,N-dimethylethan-1-amine and using 3-bromooxetane as the alkylating agent the title product was obtained as a light brown oil (227 mg, 47%) and used directly in the next step. ¹H NMR (400 MHz, CDClI₃) δ 7.51-7.47 (m, 2H), 7.41-7.35 (m, 2H), 7.35-7.30 (m, 1H), 7.13 (s, 1H), 7.09 (t, J=8.0 Hz, 1H), 7.00 (d, J=8.0 Hz, 1H), 6.57 (d, J=7.6 Hz, 1H), 5.53-5.45 (m, 1H), 5.19 (s, 2H), 5.12 (t, J=7.2 Hz, 2H), 5.05 (t, J=6.8 Hz, 2H), 3.09-3.04 (m, 2H), 2.63-2.57 (m, 2H), 2.15 (s, 6H).

Step 2: 3-(2-(dimethylamino)ethyl)-1-(oxetan-3-yl)-1H-indol -4-ol

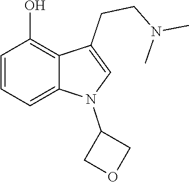 [SMILES: CN(C)CCc1cn(C2COC2)c2cccc(O)c12]

The 2-(4-(benzyloxy)-1-(oxetan-3-yl)-1H-indol -yl)-N,N-dimethylethan-1-amine from the previous step was hydrogenated following the analogous procedure to Example 6. The crude product was purified by silica gel column chromatography (eluent: DCM/MeOH/aq. NH₃ =40/1/0.5) to afford the title product as a light brown solid (73 mg, 43%). MS (ESI) calcd for C₁₅H₂₀O₂260.2; found: 261.3 [M+1]. ¹H NMR (400 MHz, CDCl₃) δ 7.11-7.06 (m, 2H), 6.84 (d, J=8.0 Hz, 1H), 6.58 (d, J=7.6 Hz, 1H), 5.53-5.45 (m, 1H), 5.12 (t, J=7.2 Hz, 2H), 5.03 (t, J=6.8 Hz, 1H), 2.99-2.94 (m, 2H), 2.74-2.70 (m, 2H), 2.39 (s, 6H).

Example 11: 1-(but-3-en-1-yl)-3-(2-(dimethylamino)ethyl)-1H-indol-4-ol

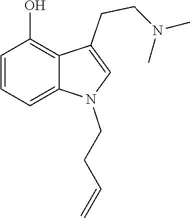 [SMILES: C=CCCn1cc(CCN(C)C)c2c(O)cccc21]

Step 1: 2-(4-(benzyloxy)-1-(but-3-en-1-yl)-1H-indol-3-yl)-N,N-dimethylethan-1-amine

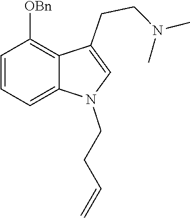 [SMILES: C=CCCn1cc(CCN(C)C)c2c(OCc3ccccc3)cccc21]

Following the analogous procedure, used in Example 6 starting from the intermediate 2-(4-(benzyloxy)-1H-indol-3-yl)-N,N-dimethylethan-1-amine and using but-3-en-1yl methanesulfonate as the alkylating agent the title product was obtained as a light brown oil (345 mg, 32%) and used directly in the next step. ¹H NMR (400 MHz, CDCl₃) δ 7.52-7.48 (m, 2H), 7.41-7.35 (m, 2H), 7.34-7.29 (m, 1H), 7.07 (t, J=8.0 Hz, 1H), 6.92 (d, J=8.0 Hz, 1H), 6.79 (s, 1H), 6.53 (d, J=7.6 Hz, 1H), 5.83-5.73 (m, 1H), 5.18 (s, 2H), 5.12-5.02 (m, 2H), 4.08 (t, J=7.4 Hz, 2H), 3.06-3.00 (m, 2H), 2.60-2.50 (m, 4H), 2.13 (s, 6H).

Step 2: 1-(but-3-en-1-yl)-3-(2-(dimethylamino)ethyl)-1H-indol-4-ol

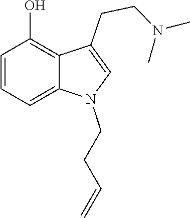 [SMILES: C=CCCn1cc(CCN(C)C)c2c(O)cccc21]

To a solution of 2-(4-(benzyloxy)-1-(but-3-en-1-yl)-1H-indol-3-yl)-N,N-dimethylethan-1-amine (392 mg, 1.12 mmol) in dried DCM (5 mL) under dry ice-EtOH bath cooling was added dropwise BBr₃ solution in DCM (1.7 mL, 1M, 1.7 mmol). After stirring at that temperature for 2 h, the reaction was slowly quenched with methanol. The resulting mixture was neutralized with aqueous NaHCO₃ and then extracted with EtOAc. The organic phase was separated, washed with brine, dried over Na₂SO₄, and concentrated under reduced pressure. The concentrate was purified by silica gel column chromatography (eluent: DCM/MeOH/aq. NH₃=50/1/0.5) to afford the title product as a colorless oil (92 mg, 31%). MS (ESI) calcd for C₁₆H₂₂N₂O 258.2; found: 259.4 [M+1]. ¹H NMR (400 MHz, CDCl₃) δ 7.07 (t, J=8.0 Hz, 1H), 6.80 (d, J=8.0 Hz, 1H), 6.72 (s, 1H), 6.54 (d, J=7.6 Hz, 1H), 5.84-5.73 (m, 1H), 5.12-5.03 (m, 2H), 4.05 (t, J=7.4 Hz, 2H), 2.94-2.90 (m, 2H), 2.70-2.66 (m, 2H), 2.58-2.51 (m, 2H), 2.37 (s, 6H).

Example 12: 3-(2-(ethyl(methyl)amino)ethyl)-1-methyl-1H-indol -4-ol hydrochloride

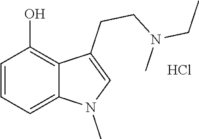 [SMILES: CCN(C)CCc1cn(C)c2cccc(O)c12 ; Cl]

Step 1: 2-(4-(benzyloxy)-1H-indol-3-yl)-N-ethyl-N-methyl-2-oxoacetamide

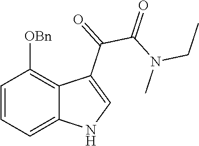 [SMILES: CCN(C)C(=O)C(=O)c1c[nH]c2cccc(OCc3ccccc3)c12]

To a stirred solution of 4-(benzyloxy)-1H-indole (3.0 g, 13 mmol) in diethyl ether (100 mL) under ice-salt bath cooling was added a solution of oxalyl chloride (3.4 g, 27 mmol) in ether dropwise.Tthe resulting brown solution was stirred for 3 h, and then added dropwise into a solution of methylethyl amine (4.0 g, 67 mmol) in ether under ice-salt bath cooling. The resulting mixture was diluted with DCM (10 mL) and then allowed to warmup to room temperature. After stirring overnight, the mixture was filtered. The filter cake was dissolved in DCM, washed with brine, dried over Na₂SO₄, and concentrated under reduced pressure. The residue was purified by silica gel column chromatography (eluent: PE/EtOAc=1/1) to afford the title compound as a brown solid (2.7 g, 60%) which was used without further purification in the next step.

Step 2: 2-(4-(benzyloxy)-1H-indol-3-yl)-N-ethyl-N-methylethan-1-amine

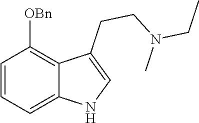 [SMILES: CCN(C)CCc1c[nH]c2cccc(OCc3ccccc3)c12]

To a stirred suspension of LiAlH₄ (765 mg, 20.1 mmol) in anhydrous THF/dioxane (1/2, 30 mL) at 60° C. under nitrogen atmosphere was added a solution of 2-(4-(benzyloxy)-1H-indol-3-yl)-N-ethyl-N-methyl-2-oxoacetamide (1.32 g, 3.87 mmol) in anhydrous THF/dioxane (1/2, 45 mL) dropwise. The mixture was stirred for 2 h at 70° C., and then heated to 105° C. overnight. The mixture was cooled down to toom temperature, and then quenched with aqueous THF under ice-water bath cooling. The mixture was diluted with THF, and then filtered through celite. The filter cake was rinsed with THF several times. The combined filtrates were concentrated under reduced pressure, and the residue purified by by silica gel column chromatography (eluent: DCM/MeOH/aq. NH3=30/1/0.5) to afford the title compound as a light brown solid (875 mg, 73%). ¹H NMR (400 MHz, CDCl₃) δ 8.03 (s, 1H), 7.52-7.48 (m, 2H), 7.40-7.35 (m, 2H), 7.33-7.29 (m, 1H), 7.05 (t, J=8.0 Hz, 1H), 6.96 (d, J=8.4 Hz, 1H), 6.89 (s, 1H), 6.54 (d, J=8.0 Hz, 1H), 5.19 (s, 2H), 3.09-3.03 (m, 2H), 2.71-2.65 (m, 2H), 2.34 (q, J=7.2 Hz, 2H), 2.13 (s, 3H), 0.98 (t, J=7.2 Hz, 3H).

Step 3: 2-(4-(benzyloxy)-1-methyl-1H-indol-3-yl)-N-ethyl-N-methylethan-1-amine

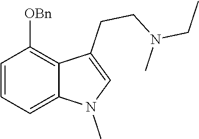 [SMILES: CCN(C)CCc1cn(C)c2cccc(OCc3ccccc3)c12]

To a stirred solution of 2-(4-(benzyloxy)-1H-indol-3-yl)-N-ethyl-N-methylethan-1-amine (1.0 g, 3.3 mmol) in DMF (20 mL) under ice-water bath cooling was added NaH (60%, 195 mg, 4.87 mmol). The resulting mixture was stirred under ice-water bath cooling for 45min, and then a solution of Mel (484 mg, 3.41 mmol) in DMF was added. The resulting mixture was stirred for an additional 2 h, and then the reaction was quenched with ice-water. The mixture was partitioned between EtOAc and water. The organic phase was washed with water and brine, dried over Na₂SO₄ and concentrated under reduced pressure. The concentrate was purified by by silica gel column chromatography (eluent: DCM/MeOH/aq. NH₃=30/1/0.5) to afford the title product as a light brown solid (719 mg, 57%) that was used directly in the next step.

Step 4: 3-(2-(ethyl(methyl)amino)ethyl)-1-methyl-1H-indol-4-ol

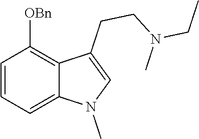 [SMILES: CCN(C)CCc1cn(C)c2cccc(OCc3ccccc3)c12]

To a stirred solution of 2-(4-(benzyloxy)-1-methyl-1H-indol-3-yl)-N-ethyl-N-methylethan-1-amine (863 mg, 2.68 mmol) in methanol (11 mL) was added 10% Pd(OH)₂/C (250 mg) and 10% Pd/C (250 mg). The resulting suspension was degassed three times under hydrogen atmosphere, and then stirred under balloon pressure hydrogen atmosphere. After stirring for 4 h, the mixture was filtered, and the filtrate was concentrated under reduced pressure. The concentrate was purified by silica gel column chromatography (eluent: DCM/MeOH/aq. NH₃=30/1/0.5) to afford the title product as a light yellow oil (167 mg, 26%). ¹H NMR (400 MHz, CDCl₃) δ 7.08 (t, J=8.0 Hz, 1H), 6.77 (d, J=8.0 Hz, 1H), 6.68 (s, 1H), 6.55 (d, J=7.6 Hz, 1H), 3.67 (s, 3H), 2.96-2.91 (m, 2H), 2.73-2.69 (m, 2H), 2.57 (q, J=7.2 Hz, 2H), 2.37 (s, 3H), 1.06 (t, J=7.2 Hz, 3H).

Step 5: 3-(2-(ethyl(methyl)amino)ethyl)-1-methyl-1H-indol-4-ol hydrochloride

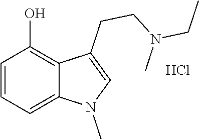 [SMILES: CCN(C)CCc1cn(C)c2cccc(O)c12 ; Cl]

To a stirred solution of 3-(2-(ethyl(methy)amino)ethyl)-1-methyl-1H-indol-4-ol (110 mg, 0.45 mmol) in DCM (3 mL) at room temperature was added 1M ethanolic HCl (0.67 mL, 0.67 mmol). The resulting solution was stirred at room temperature for 30 min, and then concentrated under reduced pressure. The concentrate was triturated with ether to afford the product as an off-white solid (96 mg, 82%). MS (ESI) calcd for C₁₄H₂₀N₂O: 232.2; found: 233.1 [M+1].¹H NMR (400 MHz, CD₃OD) δ 7.02-6.97 (m, 2H), 6.85 (d, J=8.4 Hz, 1H), 6.43 (d, J=7.4 Hz, 1H), 3.72 (s, 3H), 3.66-3.60 (m, 1H), 3.42-3.35 (m, 1H), 3.32-3.15 (m, 4H), 2.92 (s, 3H), 1.35 (t, J=7.4 Hz, 3H).

Example 13: 3-(2-(isopropyl(methy)amino)ethyl)-1-methyl-1H-indol-4-ol hydrochloride

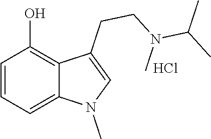 [SMILES: CC(C)N(C)CCc1cn(C)c2cccc(O)c12 ; Cl]

Step 1: 2-(4-(benzyloxy)-1H-indol-3-yl)-N-methyl-2-oxoacetamide

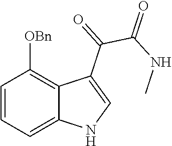 [SMILES: CNC(=O)C(=O)c1c[nH]c2cccc(OCc3ccccc3)c12]

To a solution of 4-(benzyloxy)-1H-indole (12.0 g, 53.7 mmol) in diethyl ether (400 mL) under ice-salt bath cooling was added a solution of oxalyl chloride (13.6 g, 108 mmol) in ether dropwise. The resulting brown solution was stirred for 3 h, and then added dropwise into 40% aqueous methylamine (70 mL) under ice-salt bath cooling. The resulting mixture was diluted with DCM (40 mL) and then allowed to warm up to room temperature. After stirring overnight, the mixture was concentrated under reduced pressure. The concentrate was dissolved in DCM, washed with brine, dried over Na₂SO₄, and concentrated under reduced pressure. The concentrate was purified by silica gel column chromatography (eluent: DCM/MeOH=30/1) to afford the title compound as a brown solid (7.7 g, 46%) and used directly in the next step.

Step 2: 2-(4-(benzyloxy)-1H-indol-3-yl)-N-methylethan-1-amine

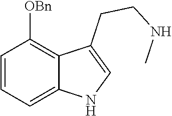 [SMILES: CNCCc1c[nH]c2cccc(OCc3ccccc3)c12]

To a stirred solution of 2-(4-(benzyloxy)-1H-indol-3-yl)-N-methyl-2-oxoacetamide (10 g, 32.4 mmol) in anhydrous THF (160 mL) under ice-water bath cooling and nitrogen atmosphere was added 1M borane-THF complex (98 mL, 98 mmol) dropwise. The resulting mixture was wheated at 70° C. overnight. The reaction was quenched slowly with 1N HCl under ice-water bath cooling. The mixture was stirred for 2 h at room temperature, and then adjusted to basic pH with ammonia under ice-water bath cooling. The aqueous phase was extracted with DCM/MeOH (10/1). The combined organic phases were washed with brine, dried over Na₂SO₄, and concentrated under reduced pressure. The crude product was purified by silica gel column chromatography (eluent: DCM/MeOH/aq. NH₃=40/1/0.5 to 10/1/0.5 gradient) to afford the title product as a light brown oil (2.6 g, 28%) that was used directly in the next step.

Step 3: N-(2-(4-(benzyloxy)-1H-indol-3-yl)ethyl)-N-methylpropan-2-amine

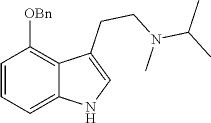 [SMILES: CC(C)N(C)CCc1c[nH]c2cccc(OCc3ccccc3)c12]

A mixture 2-(4-(benzyloxy)-1H-indol-3-yl)-N-methylethan-1-amine (600 mg, 2.14 mmol), 2-iodopropane (716 mg, 4.29 mmol), and DI PEA (555 mg, 4.29 mmol) in DMF (6 mL) was heated to 80° C. under nitrogen atmosphere. After stirring overnight, the mixture was partitioned between EtOAc and water. The organic phase was washed with water and brine, dried over Na₂SO₄ and concentrated under reduced pressure. The concentrate was purified by silica gel column chromatography (eluent: DCM/MeOH/aq. NH₃=40/1/0.5) to afford the title product as a brown oil (170 mg, 24%). ¹H NMR (400 MHz, CDCl₃) δ 7.98 (s, 1H), 7.52-7.47 (m, 2H), 7.40-7.35 (m, 2H), 7.34-7.29 (m, 1H), 7.05 (t, J=8.0 Hz, 1H), 6.97 (d, J=8.0 Hz, 1H), 6.93-6.90 (m, 1H), 6.54 (d, J=7.6 Hz, 1H), 5.20 (s, 2H), 3.09-3.03 (m, 2H), 2.84-2.76 (m, 1H), 2.73-2.67 (m, 2H), 2.10 (s, 3H), 0.93 (d, J=6.4 Hz, 6H).

Step 4: N-(2-(4-(benzyloxy)-1-methyl-1H-indol-3-yl)ethyl)-N-methylpropan-2-amine

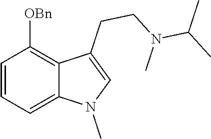 [SMILES: CC(C)N(C)CCc1cn(C)c2cccc(OCc3ccccc3)c12]

To an ice bath cooled stirred solution of N-(2-(4-(benzyloxy)-1H-indol-3-yl)ethyl)-N-methylpropan-2-amine (367 mg, 1.14 mmol) in dried THF (4 mL) under nitrogen atmosphere was added t-BuOK (154 mg, 1.37 mmol). After stirring for 30 min, a solution of Mel (195 mg, 1.37 mmol) in THF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: DCM/MeOH/aq. NH₃=40/1/0.5) to afford the title product as a light brown oil (110 mg, 23%). ¹H NMR (400 MHz, CDCl₃) δ 7.52-7.47 (m, 2H), 7.41-7.32 (m, 3H), 7.10 (t, J=8.0 Hz, 1H), 6.91 (d, J=8.0 Hz, 1H), 6.82 (s, 1H), 6.56 (d, J=8.0 Hz, 1H), 5.18 (s, 2H), 3.71 (s, 3H), 3.14-3.07 (m, 2H), 2.96-2.88 (m, 1H), 2.79-2.72 (m, 2H), 2.08 (s, 3H), 0.97 (d, J=6.8 Hz, 6H).

Step 5: 3-(2-(isopropyl(methyl)amino)ethyl)-1-methyl-1H-indol-4-ol

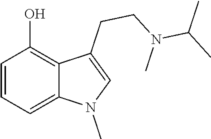 [SMILES: CC(C)N(C)CCc1cn(C)c2cccc(O)c12]

A suspension of N-(2-(4-(benzyloxy)-1-methyl-1H-indol-3-yl)ethyl)-N-methylpropan-2-amine (250 mg, 0.74 mmol), 10% Pd/C (50 mg) and 10% Pd(OH)₂/C (50 mg) in methanol (12 mL) was stirred under hydrogen atmosphere. After the starting material was consumed, the suspension was filtered. The filtrate was concentrated, and the residue purified by silica gel column chromatography (eluent: DCM/MeOH/aq. NH₃=30/1/0.5) to afford the title product as a light green oil (98 mg, 53%). ¹H NMR (400 MHz, CDCl₃) δ 7.07 (t, J=8.0 Hz, 1H), 6.78 (d, J=8.4 Hz, 1H), 6.70 (s, 1H), 6.55 (d, J=7.6 Hz, 1H), 3.67 (s, 3H), 3.03-2.95 (m, 3H), 2.80-2.75 (m, 2H), 2.40 (s, 3H), 1.04 (d, J=6.4 Hz, 6H).

Step 6: 3-(2-(isopropyl(methyl)amino)ethyl)-1-methyl-1H-indol-4-ol hydrochloride

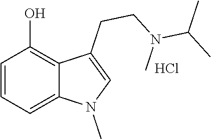 [SMILES: CC(C)N(C)CCc1cn(C)c2cccc(O)c12 ; Cl]

To a solution of 3-(2-(isopropyl(methyl)amino)ethyl)-1-methyl-1H-indol-4-ol (98 mg, 0.4 mmol) in DCM (1 mL) at room temperature was added 1M ethanolic HCl (0.6 mL, 0.6 mmol). The resulting solution was stirred at room temperature for 30 min, and then concentrated under reduced pressure. The concentrate was triturated with ether to afford the product as a white solid (91 mg, 81%). MS (ESI) calcd for C₁₆H₂₂N₂O: 246.2; found: 247.2 [M+1]. ¹H NMR (400 MHz, DMSO-d₆) δ 9.87 (s, 1H), 9.70 (s, 1H), 7.04 (s, 1H), 6.92 (d, J=7.8 Hz, 1H), 6.82 (d, J=8.0 Hz, 1H), 6.41 (d, J=8.0 Hz, 1H), 3.66 (s, 3H), 3.62-3.55 (m, 2H), 3.44-3.38 (m, 1H), 3.20-3.08 (m, 3H), 2.72 (d, J=4.8 Hz, 3H), 1.27 (d, J=6.8 Hz, 3H), 1.25 (d, J=6.4 Hz, 3H).

Example 14: 3-(2-(diisopropylamino)ethyl)-1-methyl-1H-indol-4-ol hydrochloride

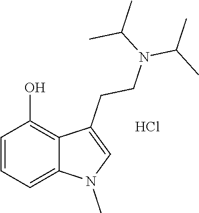 [SMILES: CC(C)N(CCc1cn(C)c2cccc(O)c12)C(C)C ; Cl]

Step 1: 2-(4-(benzyloxy)-1H-indol-3-yl)-N,N-diisopropyl-2-oxoacetamide

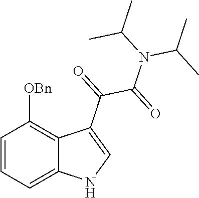 [SMILES: CC(C)N(C(=O)C(=O)c1c[nH]c2cccc(OCc3ccccc3)c12)C(C)C]

To a stirred solution of 4-(benzyloxy)-1H-indole (3.0 g, 13 mmol) in diethyl ether (65 mL) under ice-salt bath cooling was added a solution of oxalyl chloride (3.4 g, 36 mmol) in ether dropwise. The resulting brown solution was stirred for 3 h, and then added into a solution of diisopropylamine (6.78 g, 67 mmol) in ether (65 mL) under ice-salt bath cooling dropwise. The resulting mixture was diluted with DCM (10 mL) and then allowed to warmeup to room terperature. After stirring overnight, the mixture was filtered. The filter cake was dissolved in DCM, washed with brine, dried over Na₂SO₄, and concentrated under reduced pressure. The concentrate was purified by silica gel column chromatography (eluent: PE/EtOAc=1/1) to afford the title product as a yellow green solid (3.1 g, 58%) and that was used without further purification in the next step.

Step 2: N-(2-(4-(benzyloxy)-1H-indol-3-yl)ethyl)-N-isopropylpropan-2-amine

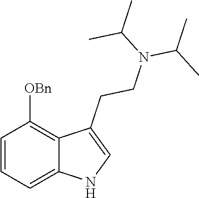 [SMILES: CC(C)N(CCc1c[nH]c2cccc(OCc3ccccc3)c12)C(C)C]

To a stirred suspension of LiAlH₄ (800 mg, 20.7 mmol) in anhydrous THF/dioxane (1/2, 30 mL) at 60° C. under nitrogen atmosphere was added a solution of 2-(4-(benzyloxy)-1H-indol -3-yl)-N,N-diisopropyl-2-oxoacetamide (1.51 g, 3.77 mmol) in anhydrous THF/dioxane (1/2, 40 mL) dropwise. The mixture was stirred for 2 h at 70° C., and then heated to 105° C. After stirring overnight, the mixture was cooled down to toom temperature, and then quenched with aqueous THF under ice-water bath cooling. The mixture was diluted with THF, and then filtered through celite. The filter cake was rinsed several times with THF. The combined filtrates were concentrated under reduced pressure, and the residue purified by silica gel column chromatography (eluent: DCM/MeOH/aq. NH₃=40/1/0.5) to afford the title product as a brown oil (1.0 g, 72%). ¹H NMR (400 MHz, CDCl₃) δ 7.96 (s, 1H), 7.48 (d, J=7.2 Hz, 2H), 7.39-7.33 (m, 2H), 7.32-7.27 (m, 1H), 7.02 (t, J=7.8 Hz, 1H), 6.95 (d, J=7.4 Hz, 1H), 6.91 (s, 1H), 6.51 (d, J=7.6 Hz, 1H), 5.23 (s, 2H), 3.07-2.97 (m, 4H), 2.76-2.68 (m, 2H), 0.96 (d, J=6.4 Hz, 12H).

Step 3: N-(2-(4-(benzyloxy)-1-methyl-1H-indol-3-yl)ethyl)-N-isopropylpropan-2-amine

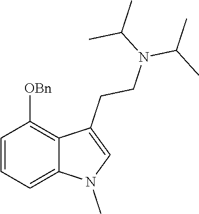 [SMILES: CC(C)N(CCc1cn(C)c2cccc(OCc3ccccc3)c12)C(C)C]

To an ice bath cooled solution of N-(2-(4-(benzyloxy)-1H-indol-3-yl)ethyl)-N-isopropylpropan-2-amine (500 mg, 1.43 mmol) in anhydrous THF (5 mL) under nitrogen atmosphere was added t-BuOK (192 mg, 1.72 mmol). After stirring for 30 min, a solution of Mel (213 mg, 1.50 mmol) in THF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (DCM/MeOH/aq. NH₃=40/1/0.5) to afford the title product as a light brown oil (350 mg, 67%) and that was used directly in the next step.

Step 4: 3-(2-(diisopropylamino)ethyl)-1-methyl-1H-indol-4-ol

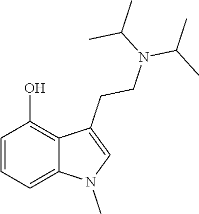 [SMILES: CC(C)N(CCc1cn(C)c2cccc(O)c12)C(C)C]

A suspension of N-(2-(4-(benzyloxy)-1-methyl-1H-indol-3-yl)ethyl)-N-isopropylpropan-2-amine (350 mg, 0.96 mmol), 10% Pd/C (40 mg) and 10% Pd(OH)₂/C (40 mg) in methanol (20 mL) was stirred under hydrogen atmosphere. After the starting material was consumed, the suspension was filtered. The filtrate was concentrated, and then purified by silica gel column chromatography (eluent: DCM/MeOH/aq. NH₃=30/1/0.5) to afford the title product as a yellow oil (168 mg, 64%). ¹H NMR (400 MHz, CDCl₃) δ 7.06 (t, J=8.0 Hz, 1H), 6.75 (d, J=8.0 Hz, 1H), 6.65 (s, 1H), 6.53 (d, J=7.6 Hz, 1H), 3.67 (s, 3H), 3.16-3.08 (m, 2H), 2.98-2.93 (m, 2H), 2.80-2.75 (m, 2H), 1.02 (d, J=6.4 Hz, 12H).

Step 5: 3-(2-(diisopropylamino)ethyl)-1-methyl-1H-indol-4-ol hydrochloride

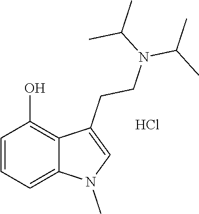 [SMILES: CC(C)N(CCc1cn(C)c2cccc(O)c12)C(C)C ; Cl]

To a solution of 3-(2-(diisopropylamino)ethyl)-1-methyl-1H-indol-4-ol (260 mg, 0.95 mmol) in DCM (4 mL) at room temperature was added 1M ethanolic HCl (1.5 mL, 1.5 mmol). The resulting solution was stirred at room temperature for 30 min, and then concentrated under reduced pressure. The concentrate was triturated with ether to afford the title product as a gray solid (208 mg, 70%). MS (ESI) calcd for C₁₇H₂₆N₂O: 274.2; found: 275.3 [M+1]. ¹H NMR (400 MHz, CD₃OD) δ 7.00 (t, J=8.0 Hz, 1H), 6.99 (s, 1H), 6.86 (d, J=8.0 Hz, 1H), 6.43 (d, J=7.6 Hz, 1H), 3.84-3.76 (m, 2H), 3.73 (s, 3H), 3.49-3.44 (m, 2H), 3.29-3.24 (m, 2H), 1.44 (m, 12H).

Example 15: 1-methyl-3-(2-(methyl(2,2,2-trifluoroethyl)amino)ethyl)-1H-indol-4-ol hydrochloride

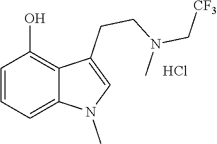 [SMILES: CN(CCc1cn(C)c2cccc(O)c12)CC(F)(F)F ; Cl]

Step 1: 2-(4-(benzyloxy)-1H-indol-3-yl)-2-oxo-N-(2,2,2-trifluoroethyl)acetamide

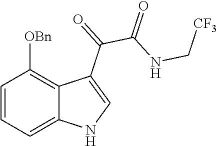 [SMILES: O=C(NCC(F)(F)F)C(=O)c1c[nH]c2cccc(OCc3ccccc3)c12]

To a stirred solution of 4-(benzyloxy)-1H-indole (3.0 g, 13 mmol) in diethyl ether (65 mL) under ice-salt bath cooling was added a solution of oxalyl chloride (3.4 g, 37 mmol) in ether dropwise.The resulting brown solution was stirred for 3 h, and then added into a solution of trifluoroethylamine (4.0 g, 40 mmol) and DIPEA (11 mL, 67 mmol) in ether (65 mL) under ice-salt bath cooling dropwise. The resulting mixture was diluted with DCM (10 mL) and then warmed up to room temperature. After stirring overnight, the mixture was partitioned between DCM and water. The organic phase was washed with brine, dried over Na₂SO₄, and concentrated under reduced pressure. The brown waxy solid crude product (5.1 g) was used directly in the next step.

Step 2: N-(2-(4-(benzyloxy)-1H-indol-3-yl)ethyl)-2,2,2-trifluoroethan-1-amine

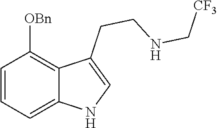 [SMILES: FC(F)(F)CNCCc1c[nH]c2cccc(OCc3ccccc3)c12]

To a stirred solution of 2-(4-(benzyloxy)-1H-indol-3-yl)-2-oxo-N-(2,2,2-trifluoroethyl)acetamide (5.0 g, 14 mmol) in anhydrous THF (72 mL) under ice-water bath cooling and nitrogen atmosphere was added 1M borane-THF complex (57 mL, 57 mmol) dropwise. The resulting mixture heated at 70° C. overnight. The reaction was quenched slowly with 1N HCl under ice-water bath cooling. The mixture was stirred for 2 h at room temperature, and then adjusted to basic pH with ammonia under ice-water bath cooling. The aqueous phase was extracted with DCM/MeOH (10/1). The combined organic phases were washed with brine, dried over Na₂SO₄, and concentrated under reduced pressure. The crude product was purified by silica gel column chromatography (eluent: PE/EtOAc=4/1) to afford the title compound as a brown oil (917 mg, 18%).¹H NMR (400 MHz, CDCl₃) δ 8.01 (s, 1H), 7.52-7.46 (m, 2H), 7.44-7.33 (m, 3H), 7.09 (t, J=8.0 Hz, 1H), 6.98 (d, J=8.0 Hz, 1H), 6.90 (s, 1H), 6.58 (d, J=7.6 Hz, 1H), 5.16 (s, 2H), 3.19 (q, J=9.6 Hz, 2H), 3.05-2.99 (m, 2H), 2.97-2.92 (m, 2H).

Step 3: tert-butyl (2-(4-(benzyloxy)-1H-indol-3-yl)ethyl)(2,2,2-trifluoroethyl)carbamate

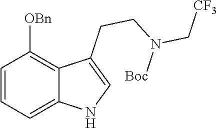 [SMILES: CC(C)(C)OC(=O)N(CCc1c[nH]c2cccc(OCc3ccccc3)c12)CC(F)(F)F]

A stirred solution of N-(2-(4-(benzyloxy)-1H-indol-3-yl)ethyl)-2,2,2-trifluoroethan-1-amine (906 mg, 2.6 mmol), Boc₂O (852 mg, 3.9 mmol) and TEA (527 mg, 5.2 mmol) in methanol (13 mL) was stirred at room temperature overnight. The solvent was evaporated under reduced pressure. The residue was purified by silica gel column chromatography (eluent: hexane/EtOAc=10/1) to afford the title compound as a light brown solid (631 mg, 54%) that was used directly in the next step.

Step 4: tert-butyl (2-(4-(benzyloxy)-1-methyl-1H-indol-3-yl)ethyl)(2,2,2-trifluoroethyl)carbamate

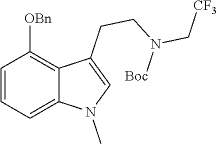 [SMILES: Cn1cc(CCN(CC(F)(F)F)C(=O)OC(C)(C)C)c2c(OCc3ccccc3)cccc21]

To an ice bath cooled solution of tert-butyl (2-(4-(benzyloxy)-1H-indol-3-yl)ethyl)(2,2,2-trifluoroethyl)carbamate (603 mg, 1.34 mmol) in DMF (3 mL) under nitrogen atmosphere was added t-BuOK (180 mg, 1.61 mmol). After stirring for 30 min, a solution of Mel (230 mg, 1.61 mmol) in DMF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: PE/EtOAc=20/1) to afford the title compound as a colorless oil (429 mg, 69%). ¹H NMR (400 MHz, CDCl₃) δ 7.50-7.45 (m, 2H), 7.43-7.38 (m, 2H), 7.37-7.32 (m, 1H), 7.15-7.08 (m, 1H), 6.91 (d, J=8.0 Hz, 1H), 6.80-6.64 (m, 1H), 6.57 (d, J=8.0 Hz, 1H), 5.17 (s, 2H), 3.70 (s, 3H), 3.50-3.44 (m, 2H), 3.38 (q, J=9.2 Hz) and 3.24 (q, J=8.8 Hz) (together 2H, approx. 1:1, 2 rotamers), 3.07-2.96 (m, 2H), 1.44 (s), 1.35 (s) (together 9H, approx. 1:1, 2 rotamers).

Step 5: N-(2-(4-(benzyloxy)-1-methyl-1H-indol-3-yl)ethyl)-2,2,2-trifluoro-N-methylethan-1-amine

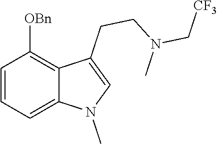 [SMILES: CN(CCc1cn(C)c2cccc(OCc3ccccc3)c12)CC(F)(F)F]

To a stirred suspension of LiAlH₄ (105 mg, 2.76 mmol) in dried THF (3 mL) under ice-water bath cooling and nitrogen atmosphere was added a solution of tert-butyl (2-(4-(benzyloxy)-1-methyl-1H-indol-3-yl)ethyl)(2,2,2-trifluoroethyl)carbamate (426 mg, 0.92 mmol) in anhydrous THF. The resulting mixture was heated to 80° C., and stirred overnight. The mixture was allowed to cool to room temperature and cautiously quenched with ice-water. The resuling mixture was filtered through celite, and the filter cake was rinsed several times with THF. The organic phase was dried, and concentrated under reduced pressure. The residue was purified by silica gel column chromatography (eluent: PE/EtOAc=10/1) to afford the title product as a light brown oil (123 mg, 35%) that was used directly in the next step.

Step 6: 1-methyl-3-(2-(methyl(2,2,2-trifluoroethyl)amino)ethyl)-1H-indol-4-ol hydrochloride

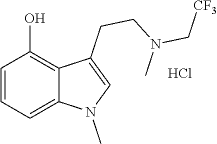 [SMILES: CN(CCc1cn(C)c2cccc(O)c12)CC(F)(F)F ; Cl]

A suspension of N-(2-(4-(benzyloxy)-1-methyl-1H-indol-3-yl)ethyl)-2,2,2-trifluoro-N-methylethan-1-amine (123 mg, 0.32 mmol), 10% Pd/C (40 mg) and 10% Pd(OH)₂/C (40 mg) in methanol (20 mL) was stirred under hydrogen atmosphere. After the starting material was consumed, the suspension was filtered. The filtrate was concentrated and the residue was purified by silica gel column chromatography (eluent: DCM/MeOH/aq. NH₃=30/1/0.5) to afford a colorless oil (63 mg, 67%). The oil was dissolved in DCM (2 mL) at room temperature and 1M ethanolic HCl (0.3 mL, 0.3 mmol) was then added. The resulting solution was stirred at room temperature for 30 min, and then concentrated under reduced pressure. The concentrate was triturated with ether to afford the title product as an off-white solid (38 mg, 56%). MS (ESI) calcd for C₁₄H₁₇F₃N₂O, Exact Mass: 286.1; found: 287.2 [M+1]. ¹H NMR (400 MHz, CD₃OD) δ 7.02-6.97 (m, 2H), 6.85 (d, J=8.4 Hz, 1H), 6.44 (d, J=7.6 Hz, 1H), 4.35 (q, J=8.8 Hz, 2H), 3.75-3.66 (m, 5H), 3.38-3.34 (m, 2H), 3.15 (s, 3H).

Example 16: 3-(2-((cyclopropylmethyl) (methyl)amino)ethyl)-1-methyl-1H-indol-4-ol hydrochloride

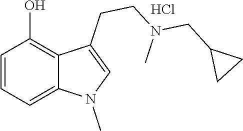 [SMILES: CN(CCc1cn(C)c2cccc(O)c12)CC1CC1 ; Cl]

Step 1: 2-(4-(benzyloxy)-1H-indol-3-yl)-N-(cyclopropylmethyl)-N-methylethan-1amine

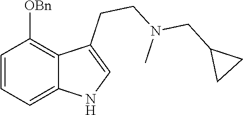 [SMILES: CN(CCc1c[nH]c2cccc(OCc3ccccc3)c12)CC1CC1]

To a stirred solution of 2-(4-(benzyloxy)-1H-indol-3-yl)-N-methylethan-1-amine (600 mg, 2.14 mmol) and cyclopropanecarboxaldehyde (450 mg, 6.43 mmol) in DCE/MeOH (10/1, 11 mL) at room temperature was added a drop of HOAc . . , NaBH(OAc)₃ (1.36 g, 6.43 mmol) was added and the resulting mixture was stirred at room temperature until the starting material was consumed. The mixture was partitioned between EtOAc and aq. Na₂CO₃. The organic phase was washed with brine, dired over Na₂SO₄, and concentrated under reduced pressure. The residue was purified by silica gel column chromatography (eluent: DCM/MeOH/aq. NH₃=30/1/0.5) to afford the title compound as a light brown oil (176 mg, 24%). ¹H NMR (400 MHz, CDCl₃) δ 8.08 (s, 1H), 7.53-7.47 (m, 2H), 7.41-7.35 (m, 2H), 7.35-7.30 (m, 1H), 7.06 (t, J=8.4 Hz, 1H), 6.97 (d, J=8.0 Hz, 1H), 6.90 (s, 1H), 6.55 (d, J=7.6 Hz, 1H), 5.17 (s, 2H), 3.12-3.05 (m, 2H), 2.81-2.75 (m, 2H), 2.25-2.18 (m, 5H), 0.90-0.80 (m, 1H), 0.49-0.44 (m, 2H), 0.08-0.04 (m, 2H).

Step 2: 2-(4-(benzyloxy)-1-methyl-1H-indol-3-yl)-N-(cyclopropylmethyl)-N-methylethan-1-amine

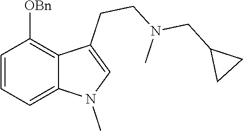 [SMILES: CN(CCc1cn(C)c2cccc(OCc3ccccc3)c12)CC1CC1]

To an ice bathcooled stirred solution of 2-(4-(benzyloxy)-1H-indol-3-yl)-N-(cyclopropylmethyl)-N-methylethan-1-amine (460 mg, 1.38 mmol) in anhydrous THF (7 mL) under nitrogen atmosphere was added t-BuOK (147 mg, 1.31 mmol). After stirring for 30 min, a solution of Mel (186 mg, 1.31 mmol) in THF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: PE/EtOAc=20/1) to afford the title compound as a light brown oil (257 mg, 53%). ¹H NMR (400 MHz, CDCl₃) δ 7.45-7.40 (m, 2H), 7.35-7.24 (m, 3H), 7.03 (t, J=8.0 Hz, 1H), 6.84 (d, J=8.0 Hz, 1H), 6.72 (s, 1H), 6.48 (d, J=8.0 Hz, 1H), 5.10 (s, 2H), 3.63 (s, 3H), 3.05-2.99 (m, 2H), 2.76-2.70 (m, 2H), 2.17-2.13 (m, 5H), 0.83-0.75 (m, 1H), 0.45-0.39 (m, 2H), 0.03-0.00 (m, 2H).

Step 3: 3-(2-((cyclopropylmethyl)(methyl)amino)ethyl)-1-methyl-1H-indol-4-ol hydrochloride

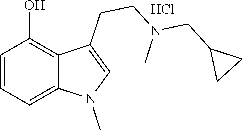 [SMILES: CN(CCc1cn(C)c2cccc(O)c12)CC1CC1 ; Cl]

A suspension of 2-(4-(benzyloxy)-1-methyl-1H-indol-3-yl)-N-(cyclopropylmethyl)-N-methylethan-1-amine (198 mg, 0.56 mmol), 10% Pd/C (40 mg) and 10% Pd(OH)₂/C (40 mg) in methanol (20 mL) was stirred under hydrogen atmosphere at room temperature. After the starting material was consumed, the suspension was filtered. The filtrate was concentrated, and the residue purified by silica gel column chromatography (eluent: DCM/MeOH/aq. NH₃=30/1/0.5) to afford the title product as a light brown oil (40 mg, 27%). The oil was dissolved in DCM (2 mL) at room temperature and 1M ethanolic HCl (0.2 mL, 0.2 mmol) was added. The resulting solution was stirred at room temperature for 30 min, and then concentrated under reduced pressure. The concentrate was triturated with ether to afford the title product as a gray solid (28 mg, 62%). MS (ESI) calcd for C₁₆H₂₂N₂O, Exact Mass: 258.2; found: 259.3 [M+1]. ¹H NMR (400 MHz, CD₃OD) δ 6.99 (t, J=8.0 Hz, 1H), 6.98 (s, 1H), 6.85 (d, J=8.0 Hz, 1H), 6.44 (d, J=7.6 Hz, 1H), 3.79-3.73 (m, 1H), 3.72 (s, 3H), 3.47-3.39 (m, 1H), 3.32-3.25 (m, 2H), 3.22-3.15 (m, 1H), 3.08-3.01 (m, 1H), 2.98 (s, 3H), 1.20-1.11 (m, 1H), 0.78-0.73 (m, 2H), 0.45-0.40 (m, 2H).

Example 17: 3-(2-((2,2-difluoroethyl) (methyDamino)ethyl)-1-methyl-1H-indol-4-ol hydrochloride

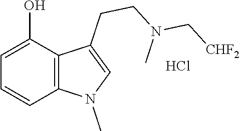 [SMILES: CN(CCc1cn(C)c2cccc(O)c12)CC(F)F ; Cl]

Step 1: 2-(4-(benzyloxy)-1H-indol-3-yl)-N-(2,2-difluoroethyl)-2-oxoacetamide

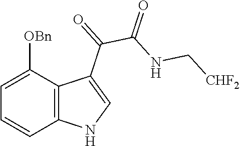 [SMILES: O=C(NCC(F)F)C(=O)c1c[nH]c2cccc(OCc3ccccc3)c12]

To a stirred solution of 4-(benzyloxy)-1H-indole (5.0 g, 22 mmol) in diethyl ether (170 mL) under ice-salt bath cooling was added a solution of oxalyl chloride (5.67 g, 45 mmol) in ether dropwise. The resulting brown solution was stirred for 3 h, and then added into a solution of 2,2-difluoroethan-1-amine (9.0 g, 110 mmol) and DIPEA (18.5 mL) in ether (110 mL) under ice-salt bath cooling dropwise. The resulting mixture was diluted with DCM (20 mL) and then allowed to warm up to room terperature. After stirring overnight, the mixture was partitioned between DCM and water. The organic phase was washed with brine, dried over Na₂SO₄, and concentrated under reduced pressure. The crude brown wax-like solid product (8.5 g) was used directly in the next step.

Step 2: N-(2-(4-(benzyloxy)-1H-indol-3-ypethyl)-2,2-difluoroethan-1-amine

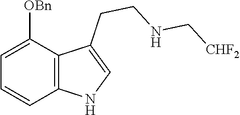 [SMILES: FC(F)CNCCc1c[nH]c2cccc(OCc3ccccc3)c12]

To a stirred solution of 2-(4-(benzyloxy)-1H-indol-3-yl)-N-(2,2-difluoroethyl)-2-oxoacetamide (3.0 g, 9.11 mmol) in anhydrous THF (45 mL) under ice-water bath cooling and nitrogen atmosphere was added 1M borane-THF complex (37 mL, 37 mmol) dropwise. The resulting mixture was heated at 70° C. overnight. The starting material was consumed, and the reaction was quenched slowly with 1N HCl under ice-water bath cooling. The mixture was stirred for 2 h at room temperature, and then adjusted to basic pH with ammonia under ice-water bath cooling. The aqueous phase was extracted with DCM/MeOH (10/1). The combined organic phases were washed with brine, dried over Na₂SO₄, and concentrated under reduced pressure. The crude product residue was purified by silica gel column chromatography (eluent: PE/EtOAc=4/1) to afford the title compound as a brown oil which was directly used in the next step without further purification.

Step 3: tert-butyl (2-(4-(benzyloxy)-1H-indol-3-yl)ethyl)(2,2-difluoroethyl)carbamate

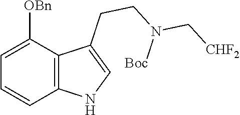 [SMILES: CC(C)(C)OC(=O)N(CCc1c[nH]c2cccc(OCc3ccccc3)c12)CC(F)F]

A solution of N-(2-(4-(benzyloxy)-1H-indol-3-ypethyl)-2,2-difluoroethan-1-amine from above (1.2 g), Boc₂O (1.19 mg, 5.45 mmol) and TEA (736 mg, 7.27 mmol) in methanol (18 mL) was stirred at room temperature overnight. The solvent was evaporated under reduced pressure. The residue was purified by silica gel column chromatography (eluent: PE/EtOAc=10/1) to afford the title compound as a light brown solid (322 mg, 9% over three steps) which was used directly in the next step.

Step 4: tert-butyl (2-(4-(benzyloxy)-1-methyl-1H-indol-3-yl)ethyl)(2,2-difluoroethyl)carbamate

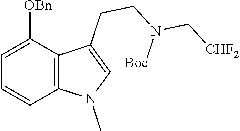 [SMILES: Cn1cc(CCN(CC(F)F)C(=O)OC(C)(C)C)c2c(OCc3ccccc3)cccc21]

To an ice bath cooled stirred solution of tert-butyl (2-(4-(benzyloxy)-1H-indol-3-yl)ethyl)(2,2-difluoroethyl)carbamate (220 mg, 0.51 mmol) in DMF (3 mL) under nitrogen atmosphere was added NaH (60%, 31 mg, 0.77 mmol). After stirring for 30 min, a solution of Mel (77 mg, 0.54 mmol) in DMF was added. After the starting material was consumed, the reaction mixture was quenched with water, and then diluted with EtOAc. The organic phase was washed with water and brine, dried over Na₂SO₄ and concentrated under reduced pressure. The concentrate was purified by silica gel column chromatography (eluent: PE/EtOAc=10/1) to afford the title compound as a brown oil (234 mg, 79%) which was used directly in the next step.

Step 5: N-(2-(4-(benzyloxy)-1-methyl-1H-indol-3-yl)ethyl)-2,2-difluoro-N-methylethan-1-amine

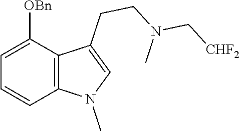 [SMILES: CN(CCc1cn(C)c2cccc(OCc3ccccc3)c12)CC(F)F]

To a stirred suspension of LiAlH₄ (53 mg, 1.55 mmol) in anhydrous THF (2 mL) under ice-water bath cooling and ntrogen atmosphere was added a solution of tert-butyl (2-(4-(benzyloxy)-1-methyl-1 H-indol-3-yl)ethyl)(2,2,2-trifluoroethyl)carbamate (230 mg, 0.51 mmol) in anhydrous THF. The resulting mixture wa heated to 80° C., and stirred overnight. The reaction was allowed to cool to room temperature and was cautiously quenched with ice-water. The mixture was filtered through ceilite, and the filter cakewas rinsed several times with THF. The combined organic phases were dried over sodium sulfate, and concentrated under reduced pressure. The residue was purified by silica gel column chromatography (eluent: PE/EtOAc=10/1) to afford the title compound as a light brown oil (102 mg, 55%) which was used directly in the next step.

Step 6: 3-(2-((2,2-difluoroethyl)(methyl)amino)ethyl)-1-methyl-1H-indol-4-ol hydrochloride

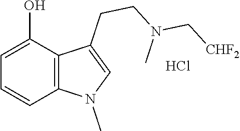 [SMILES: CN(CCc1cn(C)c2cccc(O)c12)CC(F)F ; Cl]

A suspension of N-(2-(4-(benzyloxy)-1-methyl-1 H-indol-3-yl)ethyl)-2,2-difluoro-N-methylethan-1-amine (100 mg, 0.27 mmol), 10% Pd/C (20 mg) and 10% Pd(OH)₂/C (20 mg) in methanol (20 mL) was stirred under hydrogen atmosphere. After the starting material was consumed, the suspension was filtered. The filtrate was concentrated, and the residue was purified by silica gel column chromatography (eluent: DCM/MeOH/aq. NH₃=30/1/0.5) to afford a light brown oil (54 mg, 72%). The oil was dissolved in DCM (2 mL) at room temperature and 1M ethanolic HCl (0.2 mL, 0.2 mmol) was then added. The resulting solution was stirred at room temperature for 30 min, and then concentrated under reduced pressure. The concentrate was triturated with ether to afford the salt as an off-white solid (38 mg, 67%). MS (ESI) calcd for C₁₄H₁₈F₂N₂O, Exact Mass: 268.1; found: 269.2 [M+1]. ¹H NMR (400 MHz, CD₃OD) δ 7.02-6.97 (m, 2H), 6.85 (d, J=8.0 Hz, 1H), 6.47 (tt, J=53.6, 3.6 Hz, 1H), 6.44 (d, J=7.6 Hz, 1H), 3.65-3.52 (m, 7H), 3.10 (s, 3H), 3.36-3.30 (m, 2H).

Example 18: 3-(2-(diethylamino)ethyl)-1-methyl-1H-indol-4-ol hydrochloride

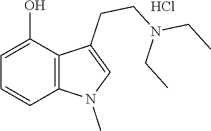 [SMILES: CCN(CC)CCc1cn(C)c2cccc(O)c12 ; Cl]

Step 1: 2-(4-(benzyloxy)-1H-indol-3-yl)-N,N-diethyl-2-oxoacetamide

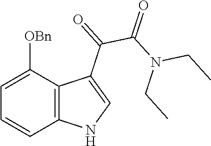 [SMILES: CCN(CC)C(=O)C(=O)c1c[nH]c2cccc(OCc3ccccc3)c12]

To a stirred solution of 4-(benzyloxy)-1H-indole (5.0 g, 22 mmol) in diethyl ether (170 mL) under ice-salt bath cooling was added a solution of oxalyl chloride (5.68 g, 44.8 mmol) in ether dropwise. The resulting brown solution was stirred for 3 h, and then added into a solution of diethylamine (8.2 g, 112 mmol) in ether (110 mL) under ice-salt bath cooling dropwise. The resulting mixture was diluted with DCM (10 mL) and then allowed to warm up to room terperature. After stirring overnight, the mixture was filtered. The filter cake was dissolved in DCM, washed with brine, dried over Na₂SO₄, and concentrated under reduced pressure. The concentrate was purified by silica gel column chromatography (eluent: PE/EtOAc=1/1) to afford the title compound as a light brown solid (2.7g, 34%) which was used directly in the next step.

Step 2: 2-(4-(benzyloxy)-1H-indol-3-yl)-N,N-diethylethan-1-amine

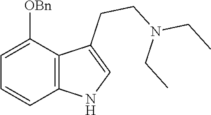 [SMILES: CCN(CC)CCc1c[nH]c2cccc(OCc3ccccc3)c12]

To a stirred suspension of LiAlH₄ (734 mg, 19.3 mmol) in anhydrous THF/dioxane (1/2, 30 mL) at 60° C. under nitrogen atmosphere was added a solution of 2-(4-(benzyloxy)-1H-indol-3-yl)-N,N-diethyl-2-oxoacetamide (1.3 g, 3.7 mmol) in anhydrous THF/dioxane (1/2, 40 mL) dropwise. The mixture was stirred for 2 h at 70° C., and then heated to 105° C. After stirring overnight, the mixture was cooled down to room temperature, and then quenched with aqueous THF under ice-water bath cooling. The mixture was diluted with THF, and then filtered through celite. The filter cake was rinsed several times with THF. The combined filtrates were concentrated under reduced pressure, and the residue purified by silica gel column chromatography (eluent: DCM/MeOH/aq. NH₃=30/1/0.5) to afford the title compound as a light yellow solid (876 mg, 73%) which was used directly in the next step.

Step 3: 2-(4-(benzyloxy)-1-methyl-1H-indol-3-yl)-N,N-diethylethan-1-amine

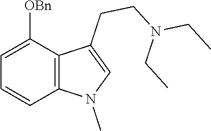 [SMILES: CCN(CC)CCc1cn(C)c2cccc(OCc3ccccc3)c12]

To a stirred ice bath cooled solution of 2-(4-(benzyloxy)-1H-indol-3-yl)-N,N-diethylethan-1-amine (620 mg, 2.45 mmol) in DMF (10 mL) under nitrogen atmosphere was added NaH (60%, 116 mg, 2.89 mmol). After stirring for 30 min, a solution of Mel (287 mg, 2.02 mmol) in DMF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: DCM/MeOH/aq. NH₃=30/1/0.5) to afford the title product as brown oil (471 mg, 73%) which was used directly in the next step.

Step 4: 3-(2-(diethylamino)ethyl)-1-methyl-1H-indol-4-ol hydrochloride

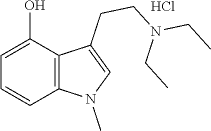 [SMILES: CCN(CC)CCc1cn(C)c2cccc(O)c12 ; Cl]

A suspension of 2-(4-(benzyloxy)-1-methyl-1H-indol-3-yl)-N,N-diethylethan-1-amine (827 mg, 2.45 mmol), 10% Pd/C (40 mg) and 10% Pd(OH)₂/C (40 mg) in methanol (20 mL) was stirred under hydrogen atmosphere. After the starting material was consumed, the suspension was filtered. The filtrate was concentrated, and then purified by silica gel column chromatography (eluent: DCM/MeOH/aq. NH₃=30/1/0.5) to afford a light brown oil (317 mg, 52%). The oil was dissolved in DCM (3 mL) at room temperature and 1M ethanolic HCl (0.9 mL, 0.9 mmol) was added. The resulting solution was stirred at room temperature for 30 min, and concentrated under reduced pressure. The concentrate was triturated with ether to afford the title product as a light purple solid (139 mg, 82%). MS (ESI) calcd for C₁₅H₂₂N₂O, Exact Mass: 246.2; found: 247.2 [M+1]. ¹H NMR (400 MHz, CD₃OD) δ 7.00 (t, J=8.0 Hz, 1H), 6.98 (s, 1H), 6.85 (d, J=8.4 Hz, 1H), 6.43 (d, J=7.6 Hz, 1H), 3.72 (s, 3H), 3.54-3.48 (m, 2H), 3.32-3.23 (m, 6H), 1.37 (t, J=7.2 Hz, 6H).

Example 19: 3-(2-(Dimethylamino)ethyl)-1-(methyl-d₃)-1H-indol-4-ol

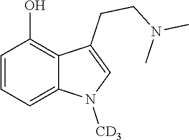 [SMILES: [2H]C([2H])([2H])n1cc(CCN(C)C)c2c(O)cccc21]

Step 1: 2-(4-(Benzyloxy)-1H-indol-3-yl)-N,N-dimethyl-2-oxoacetamide

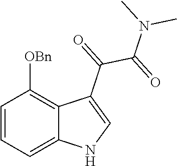 [SMILES: CN(C)C(=O)C(=O)c1c[nH]c2cccc(OCc3ccccc3)c12]

To a stirred solution of 4-(benzyloxy)-1H-indole (1.50 g, 6.73 mmol) and phthalimide (600 mg) in anhydrous diethyl ether (30 mL) at 0° C. was added dropwise oxalyl chloride (0.63 mL, 7.40 mmol under N2 atmosphere. The resulting mixture was stirred at ambient temperature for 1 h. To this was added dimethylamine (2 N in THF, 16.8 mL, 33.6 mmol), and the resulting suspension was stirred at ambient temperature for another 16 h. The mixture was diluted with saturated NaHCO₃ solution and extracted three times with EtOAc. The organic layers were combined, washed with brine, dried over Na₂SO₄, and concentrated under vacuum. The residue was triturated with EtOAc to afford 2-[4-(benzyloxy)-1H-indol-3-yl]-N,N-dimethyl-2-oxoacetamide (960 mg, 44%) as a light-yellow solid. MS (ESI) calcd for C₁₉H₁₈N₂O₃: 322; found: 323 [M+1]. ¹H NMR (300 MHz, DMSO-d₆) δ 12.27 (br s, 1H), 8.07 (s, 1H), 7.66-7.56 (m, 2H), 7.45-7.31 (m, 2H), 7.31-7.22 (m, 1H), 7.17-7.02 (m, 2H), 6.69 (dd, J=6.6, 2.4 Hz, 1H), 5.26 (s, 2 H), 2.92 (s, 3H), 2.89 (s, 3H).

Step 2: 2-(4-(Benzyloxy)-1-(methyl-d₃)-1H-indol-3-yl)-N,N-dimethyl-2-oxoacetamide

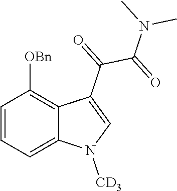 [SMILES: [2H]C([2H])([2H])n1cc(C(=O)C(=O)N(C)C)c2c(OCc3ccccc3)cccc21]

To a stirred solution of 2-[4-(benzyloxy)-1H-indol-3-yl]-N,N-dimethyl-2-oxoacetamide (500 mg, 1.55 mmol) in anhydrous DMF (20 mL) at 0° C. was added sodium hydride (60% in mineral oil, 68 mg, 1.71 mmol, 1.10 equiv) under N₂ atmosphere. The resulting mixture was stirred at ambient temperature for 20 min, then iodomethane-d₃ (248 mg, 1.71 mmol) was added dropwise. The resulting mixture was stirred at ambient temperature for 1 h and then quenched with saturated NH₄Cl solution and extracted three times with EtOAc. The combined organic layers were washed with brine, dried over Na₂SO₄, and concentrated under vacuum to afford 2-[4-(benzyloxy)-1-(methyl-d₃)-1H-indol-3-yl]-N,N-dimethyl-2-oxoacetamide (870 mg) as a semi-solid, which was used for the next step without further purification. MS (ESI) calcd for C₂₀H₁₇D₃N₂O₃: 339; found: 340 [M+1]. ¹H NMR (300 MHz, DMSO-d₆) δ 8.12 (s, 1H), 7.65-7.53 (m, 2H), 7.45-7.32 (m, 2H), 7.32-7.23 (m, 1H), 7.23-7.11 (m, 2H), 6.76 (dd, J=7.5, 1.5 Hz, 1H), 5.27 (s, 2H), 2.92 (s, 3H), 2.89 (s, 3H).

Step 2: 2-(4-(Benzyloxy)-1-(methyl-d₃)-1H-indo1-3-yl)-N,N-dimethylethan-1-amine

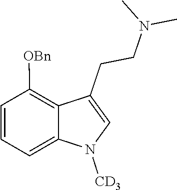 [SMILES: [2H]C([2H])([2H])n1cc(CCN(C)C)c2c(OCc3ccccc3)cccc21]

To a stirred solution of 2-[4-(benzyloxy)-1-(methyl-d₃)-1H-indol-3-yl]-N,N-dimethyl-2-oxoacetamide (850 mg, 2.51 mmol) in anhydrous THF (34 mL) at 0° C. was added dropwise borane-tetrahydrofuran complex (1M in THF, 10.0 mL, 10.0 mmol) under N₂ atmosphere. The resulting solution was stirred at ambient temperature for 16 h and then quenched with saturated NaHCO₃ solution. The mixture was stirred at ambient temperature for another 30 min and then filtered. The filtrate was extracted three times with EtOAc. The combined organic layers were washed with brine, dried over Na₂SO₄ and concentrated under vacuum. The residue was re-dissolved in ethanol (68 mL), and to the solution was added cesium fluoride (1.7 g) and sodium carbonate (1.7 g). The resulting mixture was stirred at 80° C. for another 16 h and then filtered. The filtrate was concentrated under vacuum. The residue was purified by reverse phase flash chromatography on C₁₈ silica gel (gradient eluent: 5-40% acetonitrile in water) to afford 2-[4-(benzyloxy)-1-(methyl-d₃)-1H-indol-3-yl)-N,N-dimethylethanamine (130 mg, 27% for 2 steps) as a yellow solid. MS (ESI) calcd for C₂₀H₂₁ D₃N₂O: 311; found: 312 [M+1]. ¹H NMR (300 MHz, DMSO-d₆) δ 7.57-7.48 (m, 2H), 7.45-7.26 (m, 3H), 7.06-6.90 (m, 3H), 6.57 (dd, J=7.5, 1.2 Hz, 1H), 5.17 (s, 2H), 2.96-2.84 (m, 2H), 2.48-2.40 (m, 2H), 2.06 (s, 6H).

Step 3: 3-(2-(Dimethylamino)ethyl)-1-(methyl-d₃)-1H-indol-4-ol

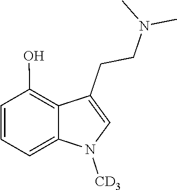 [SMILES: [2H]C([2H])([2H])n1cc(CCN(C)C)c2c(O)cccc21]

A mixture of of 2-[4-(benzyloxy)-1-(methyl-d₃)-1H-indol-3-yl]-N,N-dimethylethanamine (90 mg, 0.29 mmol) and palladium on carbon (wet, 10%, 36 mg, 25% w/w) in methanol (9 mL) was stirred at ambient temperature for 16 h under H₂ atmosphere and then filtered through a pad of celite. The filtrate was concentrated under vacuum. The residue was purified by reverse phase flash chromatography on C₁₈ silica gel (gradient eluent: 5-40% acetonitrile in water (containing 0.5% NH₄HCO₃) to afford 3-[2-(dimethylamino)ethyl]-1-(methyl-d₃)-1H-indol-4-ol (16.3 mg, 25%) as a white solid. MS (ESI) calcd for C₁₃H₁₅D₃N₂O: 221; found: 222 [M+1]. ¹H NMR (300 MHz, DMSO-d₆) δ 6.91-6.83 (m, 2H), 6.75 (dd, J=8.1, 0.9 Hz, 1H), 6.30 (dd, J=7.5, 0.9 Hz, 1H), 6.04 (br s, 1H), 2.86 (t, J=6.6 Hz, 2H), 2.55 (t, J=6.6 Hz, 2H), 2.22 (s, 6H).

Example 20: 1-methyl-3-(2-(methyl(propyl)amino)ethyl)-1H-indol-4-ol hydrochloride

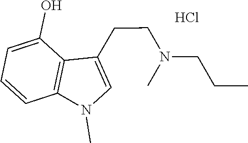 [SMILES: CCCN(C)CCc1cn(C)c2cccc(O)c12 ; Cl]

Step 1: 2-(4-(benzyloxy)-1H-indol-3-yl)-N-methyl-2-oxo-N-propylacetamide

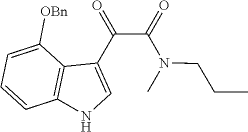 [SMILES: CCCN(C)C(=O)C(=O)c1c[nH]c2cccc(OCc3ccccc3)c12]

To a stirred solution of 4-(benzyloxy)-1H-indole (5.0 g, 22.4 mmol) in diethyl ether (170 mL) under ice-salt bath cooling was added a solution of oxalyl chloride (5.68 g, 44.8 mmol) in ether dropwise.The resulting brown solution was stirred for 3 h, and then added into a solution of N-methylpropan-1-amine (8.2 g, 112 mmol) in ether (110 mL) under ice-salt bath cooling dropwise. The resulting mixture was diluted with DCM (10 mL) and then allowed to warm up to room terperature. After stirring overnight, the mixture was filtered. The filter cake was dissolved in DCM, washed with brine, dried over Na₂SO₄, and concentrated under reduced pressure. The crude product was used directly in the next step.

Step 2: N-(2-(4-(benzyloxy)-1H-indol-3-yl)ethyl)-N-methylpropan-1-amine

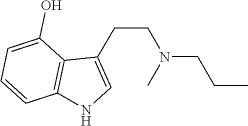 [SMILES: CCCN(C)CCc1c[nH]c2cccc(O)c12]

To a stirred suspension of LiAlH₄ (5.93 g, 156 mmol) in dried THF/dioxane (1/2, 120 mL) at 60° C. under nitrogen atmosphere was added a solution of 2-(4-(benzyloxy)-1H-indol-3-yl)-N-methyl-2-oxo-N-propylacetamide (10.6 g, crude) in dried THF/dioxane (1/2, 180 mL) dropwise.The mixture was stirred for 2 h at 70° C., heated to 105° C. overnight,cooled down to toom temperature, and then quenched with aqueous THF under ice-water bath cooling. The mixture was diluted with THF, and then filtered through celite. The filter cake was rinsed several times with THF. The combined filtrates were concentrated under reduced pressure, and the residue purified by by silica gel column chromatography (eluent: DCM/MeOH/aq. NH₃=30/1/0.5) to afford the title compound as an orange solid (4.53 g, 63% over two steps).

Step 3: N-(2-(4-(benzyloxy)-1-methyl-1H-indol-3-yl)ethyl)-N-methylpropan-1-amine

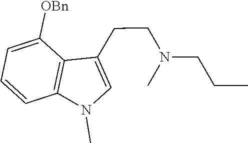 [SMILES: CCCN(C)CCc1cn(C)c2cccc(OCc3ccccc3)c12]

To an ice bath cooled stirred solution of N-(2-(4-(benzyloxy)-1H-indol-3-yl)ethyl)-N-methylpropan-1-amine (1.0 g, 3.1 mmol) in DMF (15 mL) under nitrogen atmosphere was added NaH (60%, 187 mg, 4.66 mmol). After stirring for 30 min, a solution of Mel (463 mg, 3.26 mmol) in DMF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: DCM/MeOH/aq. NH₃=30/1/0.5) to afford the title compound as brown oil (610 mg, 59%) which was used directly in the next step.

Step 4: 1-methyl-3-(2-(methyl(propyl)amino)ethyl)-1H-indol-4-ol hydrochloride

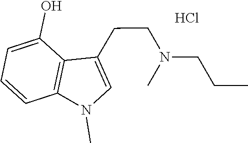 [SMILES: CCCN(C)CCc1cn(C)c2cccc(O)c12 ; Cl]

A mixture of N-(2-(4-(benzyloxy)-1-methyl-1H-indol-3-yl)ethyl)-N-methylpropan-1-amine (610 mg, 1.81 mmol), 10% Pd/C (40 mg) and 10% Pd(OH)₂/C (40 mg) in methanol (20 mL) was stirred under hydrogen atmosphere. After the starting material was consumed, the suspension was filtered. The filtrate was concentrated, and then purified by silica gel column chromatography (eluent: DCM/MeOH/aq. NH₃=30/1/0.5) to afford a light brown oil (299 mg, 67%). A portion of the oil (110 mg,(0.45 mmol) was dissolved in DCM (2 mL) at room temperature and 1M ethanolic HCl (0.7 mL, 0.7 mmol) was added. The resulting solution was stirred at room temperature for 30 min, and then concentrated under reduced pressure. The concentrate was triturated with ether to afford the product as a gray solid (110 mg, 87%). MS (ESI) calcd for C₁₅H₂₂N₂O, Exact Mass: 246.2; found: 247.2 [M+1]. ¹H NMR (400 MHz, CD₃OD) δ 7.00 (t, J=8.0 Hz, 1H), 6.98 (s, 1H), 6.85 (d, J=8.4 Hz, 1H), 6.43 (d, J=7.6 Hz, 1H), 3.72 (s, 3H), 3.70-3.62 (m, 1H), 3.43-3.35 (m, 1H), 3.32-3.25 (m, 2H), 3.25-3.18 (m, 1H), 3.12-3.04 (m, 1H), 2.94 (s, 3H), 1.83-1.72 (m, 2H), 1.01 (t, J=7.2 Hz, 3H).

Example 21: 3-(2-((cyclopropylmethyl)amino)ethyl)-1-methyl-1H-indol-4-ol hydrochloride

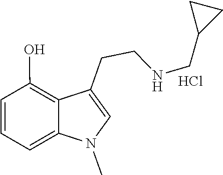 [SMILES: Cn1cc(CCNCC2CC2)c2c(O)cccc21 ; Cl]

Step 1: 2-(4-(benzyloxy)-1H-indol-3-yl)-N-(cyclopropylmethyl)-2-oxoacetamide

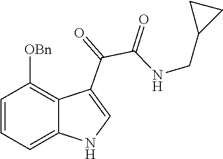 [SMILES: O=C(NCC1CC1)C(=O)c1c[nH]c2cccc(OCc3ccccc3)c12]

To a solution of 4-(benzyloxy)-1H-indole (5.0 g, 22 mmol) in diethyl ether (140 mL) under ice-salt bath cooling was added a solution of oxalyl chloride (5.7 g, 45 mmol) in ether dropwise. After completion of addition, the resulting brown solution was stirred for 3 h, and then added into a solution of cyclopropylmethanamine (8.0 g, 110 mmol) in ether (110 mL) under ice-salt bath cooling dropwise. The resulting mixture was diluted with DCM (10 mL) and then allowed to warm up to room temperature. The mixture was partitioned between DCM and water. The organic phase was washed with brine, dried over Na₂SO₄, and concentrated under reduced pressure. The light brown solid crude title compound was directly used in the next step.

Step 2: 2-(4-(benzyloxy)-1H-indol-3-yl)-N-(cyclopropylmethyl)ethan-1-amine

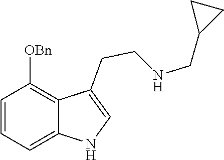 [SMILES: c1ccc(COc2cccc3[nH]cc(CCNCC4CC4)c23)cc1]

To a stirred solution of the above 2-(4-(benzyloxy)-1H-indol-3-yl)-N-(cyclopropylmethyl)-2-oxoacetamide (8.5 g) in anhydrous THF (120 mL) under ice-water bath cooling and nitrogen atmosphere was added 1M borane-THF complex (73 mL, 73 mmol) dropwise. The resulting mixture was heated at 70° C. overnight. The mixture was quenched slowly with 1N HCl under ice-water bath cooling. The mixture was stirred for 2 h at room temperature, and then adjusted to basic pH with ammonia under ice-water bath cooling. The aqueous phase was extracted with DCM/MeOH (10/1). The combined organic phases were washed with brine, dried over Na₂SO₄, and concentrated under reduced pressure. The residue was purified by silica gel column chromatography (eluent: DCM/MeOH/aq. NH₃=30/1/0.5) to afford the partially purified title compound as a brown oil which was directly used in the next step.

Step 3: tert-butyl (2-(4-(benzyloxy)-1H-indol-3-yl)ethyl)(cyclopropylmethyl)carbamate

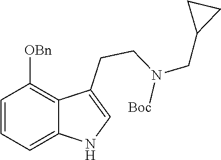 [SMILES: CC(C)(C)OC(=O)N(CCc1c[nH]c2cccc(OCc3ccccc3)c12)CC1CC1]

A solution of 2-(4-(benzyloxy)-1H-indol-3-yl)-N-(cyclopropylmethyl)ethan-1-amine (2.3 crude) and Boc₂O (1.8 g, 8.3 mmol) in methanol (18 mL) was stirred at room temperature overnight. The solvent was evaporated under reduced pressure. The residue was purified by silica gel column chromatography (eluent: PE/EtOAc=10/1) to afford the title product as a white solid (1.2 g, 25% over three steps). ¹H NMR (400 MHz, CDCl₃) δ 8.05-7.95 (m, 1H), 7.51-7.46 (m, 2H), 7.42-7.37 (m, 2H), 7.36-7.30 (m, 1H), 7.06 (t, J=8.0 Hz, 1H), 6.98 (d, J=8.0 Hz, 1H), 6.96-6.80 (m, 1H), 6.56 (d, J=7.6 Hz, 1H), 5.20 (s, 2H), 3.53-3.46 (m, 2H), 3.15- 3.05 (m, 2H), 2.90-2.68 (m, 2H), 1.45 (br) and 1.29 (br) (together 9H, 2 rotamers, approx. 1:2), 0.85-0.75 (m, 1H), 0.35-0.25 (m, 2H), 0.06-0.00 (m, 2H).

Step 4: tert-butyl (2-(4-(benzyloxy)-1-methyl-1H-indol-3-yl)ethyl)(cyclopropylmethyl)carbamate

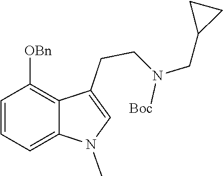 [SMILES: Cn1cc(CCN(CC2CC2)C(=O)OC(C)(C)C)c2c(OCc3ccccc3)cccc21]

To an ice bath cooled stirred solution of tert-butyl (2-(4-(benzyloxy)-1H-indol-3-yl)ethyl)(cyclopropylmethyl)carbamate (600 mg, 1.43 mmol) in DMF (6 mL) under nitrogen atmosphere was added t-BuOK (192 mg, 1.71 mmol). After stirring for 30 min, a solution of Mel (243 mg, 1.71 mmol) in DMF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: PE/EtOAc=15/1) to afford the title product as a yellow oil (510 mg, 82%) which was used directly in the next step.

Step 5: tert-butyl (cyclopropylmethyl)(2-(4-hydroxy-1-methyl-1H-indol-3-yl)ethyl)carbamate

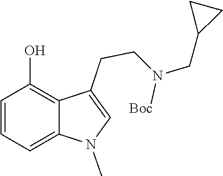 [SMILES: Cn1cc(CCN(CC2CC2)C(=O)OC(C)(C)C)c2c(O)cccc21]

A mixture of tert-butyl (2-(4-(benzyloxy)-1-methyl-1H-indol-3-yl)ethyl) (cyclopropylmethyl)carbamate (510 mg, 1.18 mmol), 10% Pd/C (100 mg) and 10% Pd(OH)₂/C (100 mg) in methanol (25 mL) was stirred under hydrogen atmosphere. After the starting material was consumed, the suspension was filtered. The filtrate was concentrated, and then purified by silica gel column chromatography (eluent: PE/EtOAc=10/1) to afford the title product as a light brown oil (245 mg, 67%) which was used directly in the next step.

Step 6: 3-(2-((cyclopropylmethylpamino)ethyl)-1-methyl-1H-indol-4-ol hydrochloride

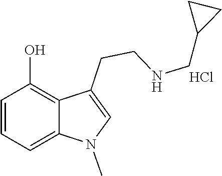 [SMILES: Cn1cc(CCNCC2CC2)c2c(O)cccc21 ; Cl]

A solution of tert-butyl (cyclopropylmethyl)(2-(4-hydroxy-1-methyl-1H-indol-3-yl)ethyl)carbamate (245 mg, 0.71 mmol) in 2M HCl/EtOH was stirred at room temperature overnight. The solvent was evaporated, and the residue was partitioned between DCM/i-PrOH (6/1) and aq. Na₂CO₃. The organic phase was washed with brine, dried over Na₂SO₄ and concentrated under reduced pressure. The concentrate was purified by silica gel column chromatography (eluent: DCM/MeOH/aq. NH₃=30/1/0.5) to afford a dark brown oil (120 mg). The oil was dissolved in DCM (2 mL) at room temperature and 1M ethanolic HCl (0.8 mL, 0.8 mmol) was added. The resulting solution was stirred at room temperature for 30 min, and then concentrated under reduced pressure. The concentrate was triturated with ether to afford the title product as a light purple solid (118 mg, 86%). MS (ESI) calcd for C₁₅H₂₀N₂O, Exact Mass: 244.2; found: 245.2 [M+1]. ¹H NMR (400 MHz, CD₃OD) δ 6.99 (t, J=8.0 Hz, 1H), 6.95 (s, 1H), 6.85 (d, J=8.0 Hz, 1H), 6.43 (d, J=7.6 Hz, 1H), 3.73 (s, 3H), 3.42 (t, J=7.2 Hz, 2H), 3.25 (t, J=7.2 Hz, 2H), 2.93 (d, J=7.6 Hz, 2H), 1.13-1.05 (m, 1H), 0.73-0.68 (m, 2H), 0.42-0.37 (m, 2H).

Example 22: 3-(2-(2,2-difluoroethyl)amino)ethyl)-1-methyl-1H-indol-4-ol hydrochloride

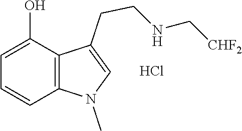 [SMILES: Cn1cc(CCNCC(F)F)c2c(O)cccc21 ; Cl]

Step 1: tert-butyl (2,2-difluoroethyl)-2-((4-hydroxy-1-methyl-1H-indol-3-yl)ethyl)carbamate

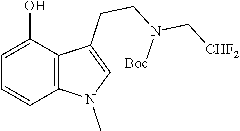 [SMILES: Cn1cc(CCN(CC(F)F)C(=O)OC(C)(C)C)c2c(O)cccc21]

A mixture of tert-butyl (2-(4-(benzyloxy)-1-methyl-1H-indol-3-yl)ethyl)(2,2-difluoroethyl)carbamate (234 mg, 0.52 mmol), 10% Pd/C (100 mg) and 10% Pd(OH)₂/C (100 mg) in methanol (15 mL) was stirred under hydrogen atmosphere. After the starting material was consumed, the suspension was filtered. The filtrate was concentrated, and then purified by silica gel column chromatography (eluent: PE/EtOAc=10/1) to afford the title compound as a light brown oil (44 mg, 23%) which was used directly in the next step.

Step 2: 3-(2-((2,2-difluoroethyl)amino)ethyl)-1-methyl-1H-indol-4-ol hydrochloride

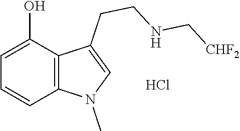 [SMILES: Cn1cc(CCNCC(F)F)c2c(O)cccc21 ; Cl]

A solution of tert-butyl (2 ,2-difl uoroethyl)(2-(4-hydroxy-1-methyl-1H-indol-3-yl)ethyl)carbamate (51 mg, 0.144 mmol) in 2M HCl/EtOH (2 mL) was stirred at room temperature until the starting material was consumed. The solvent was evaporated, and the residue was triturated with ether to afford the title product as a dark gray solid (31 mg, 74%). MS (ESI) calcd for C₁₃H₆F₂N₂O, Exact Mass: 254.1; found: 255.1 [M+1].¹H NMR (400 MHz, CD₃OD) δ 7.03-6.95 (m, 2H), 6.85 (d, J=8.4 Hz, 1H), 6.44 (d, J=7.6 Hz, 1H), 6.31 (tt, J=3.2, 53.6 Hz, 1H), 3.72 (s, 3H), 3.59 (dt, J=3.2, 15.2 Hz, 2H), 3.51 (t, J=7.6 Hz, 2H), 3.29 (t, J=7.2 Hz, 2H).

Example 23: 1-methyl-3-(2-((2,2,2-trifluoroethyl) amino)ethyl)-1H-indol-4-ol hydrochloride

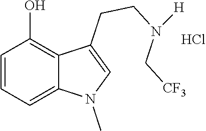 [SMILES: Cn1cc(CCNCC(F)(F)F)c2c(O)cccc21 ; Cl]

Step 1: tert-butyl (2-(4-hydroxy-1-methyl-1H-indol-3-yl)ethyl)(2,2,2-trifluoroethyl)carbamate

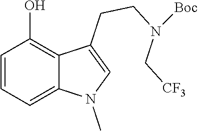 [SMILES: Cn1cc(CCN(CC(F)(F)F)C(=O)OC(C)(C)C)c2c(O)cccc21]

A mixture of tert-butyl (2-(4-(benzyloxy)-1-methyl-1H-indol-3-yl)ethyl)(2,2,2-trifluoroethyl)carbamate (400 mg, 0.81 mmol), 10% Pd/C (100 mg) and 10% Pd(OH)₂/C (100 mg) in methanol (15 mL) was stirred under hydrogen atmosphere. After the starting material was consumed, the suspension was filtered. The filtrate was concentrated, and the residue purified by silica gel column chromatography (eluent: PE/EtOAc=10/1) to afford the title compound as a white solid (280 mg, 92%) which was used directly in the next step.

Step 2: 1-methyl-3-(2-((2,2,2-trifluoroethyl)amino)ethyl)-1H-indol-4-ol hydrochloride

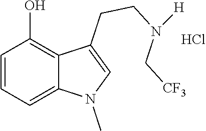 [SMILES: Cn1cc(CCNCC(F)(F)F)c2c(O)cccc21 ; Cl]

A solution of tert-butyl (2-(4-(benzyloxy)-1-methyl-1H-indol-3-yl)ethyl)(2,2,2-trifluoroethyl)carbamate (280 mg, 0.75 mmol) in 2M ehtanolic HCl (5 mL) was stirred at room temperature overnight. The solvent was evaporated, and the concentrate was triturated with ether to afford the title product as a white solid (194 mg, 84%). MS (ESI) calcd for C₁₃H₁₅F₃N₂O, Exact Mass: 272.1; found: 273.0 [M+1]. ¹H NMR (400 MHz, CD₃OD) δ 7.00 (t, J=8.0 Hz, 1H), 6.97 (s, 1H), 6.86 (d, J=8.0 Hz, 1H), 6.44 (d, J=7.6 Hz, 1H), 4.08 (q, J=8.8 Hz, 2H), 3.73 (s, 3H), 3.55 (t, J=8.0 Hz, 2H), 3.31 (t, J=7.2 Hz, 2H).

Example 24: 3-(2-((2-methoxybenzyl)amino)ethyl)-1-methyl-1H-indol-4-ol hydrochloride

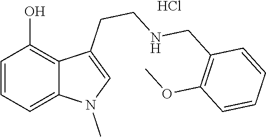 [SMILES: Cl ; COc1ccccc1CNCCc1cn(C)c2cccc(O)c12]

Step 1: 2-(4-(benzyloxy)-1H-indol-3-yl)-N-(2-methoxybenzyl)ethan-1-amine

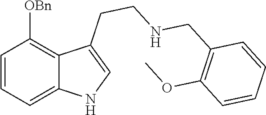 [SMILES: COc1ccccc1CNCCc1c[nH]c2cccc(OCc3ccccc3)c12]

To a stirred solution of 4-(benzyloxy)-1H-indole (5.40 g, 24.2 mmol) in diethyl ether (120 mL) under ice-salt bath cooling was dropwise added a solution of oxalyl chloride (6.15 g, 48.5 mmol) in ether. The resulting brown solution was stirred for 3 h, then dropwise added to a solution of (2-methoxyphenyl)methanamine (8.30 g, 60.6 mmol) in ether (110 mL) under ice-salt bath cooling. The resulting mixture was diluted with DCM (20 mL) and warmed up to room temperature. The mixture was partitioned between DCM and water. The organic phase was washed with brine, dried over Na₂SO₄, and concentrated under reduced pressure. The concentrate was dissolved in anhydrous THF (120 mL) and 1M borane-THF complex (87 mL, 87 mmol) was added dropwise under ice-water bath cooling. The resulting mixture was heated at 70° C. overnight. The reaction was quenched slowly with 1N HCl under ice-water bath cooling. The mixture was stirred for 2 h at room temperature, then adjusted to basic pH with ammonia. The aqueous phase was extracted with DCM/MeOH (10/1). The combined organic phases were washed with brine, dried over Na₂SO₄, and concentrated under reduced pressure. The crude product was purified by silica gel column chromatography (eluted by DCM with 3.0 M NH₃ in MeOH in the volume ratio of 25:1 to 10:1) to afford the title product as a white solid (2.6 g, 27%).

Step 2: tert-butyl (2-(4-(benzyloxy)-1H-indol-3-yl)ethyl)(2-methoxybenzyl)carbamate

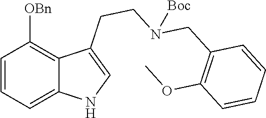 [SMILES: COc1ccccc1CN(CCc1c[nH]c2cccc(OCc3ccccc3)c12)C(=O)OC(C)(C)C]

A solution of 2-(4-(benzyloxy)-1H-indol-3-yl)-N-(2-methoxybenzyl)ethan-1-amine (2.6 g, 6.7 mmol), Boc₂O (1.9 g, 8.7 mmol) and TEA (2.0 g, 20 mmol) in methanol (30 mL) was stirred at reflux overnight. The solvent was evaporated under reduced pressure. The residue was purified by silica gel column chromatography (hexane/EtOAc=6/1˜4/1) to afford the title compound as an off-white solid (2.2 g, 67%). ¹H NMR (400 MHz, CDCl₃) δ 8.10-8.00 (m, 1H), 7.40-7.21 (m, 5H), 7.20-7.13 (m, 1H), 7.10-6.70 (m, 6H), 6.53-6.42 (m, 1H), 5.15-5.10 (m, 2H), 4.39-4.22 (m, 2H), 3.70-3.59 (m, 3H), 3.59-3.47 (m, 2H), 3.23-3.05 (m, 2H), 1.39 (s, minor rotamer) and 1.25 (s, major rotamer) (approx. 1:2; together 9H).

Step 3: tert-butyl (2-(4-(benzyloxy)-1-methyl-1H-indol-3-yl)ethyl)(2-methoxybenzyl)carbamate

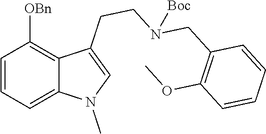 [SMILES: COc1ccccc1CN(CCc1cn(C)c2cccc(OCc3ccccc3)c12)C(=O)OC(C)(C)C]

To an ice bath cooled stirred solution of tert-butyl (2-(4-(benzyloxy)-1H-indol3 yl)ethyl)(2-methoxybenzyl)carbamate (1.2 g, 2.5 mmol) in DMF (12 mL) under nitrogen atmosphere was added t-BuOK (330 mg, 3.0 mmol). After stirring for 30 min, a solution of Mel (421 mg, 2.96 mmol) in DMF was added. After the starting material was consumed, the reaction was quenched with water, and then diluted with EtOAc. The organic phase was washed with water and brine, dried over Na₂SO₄ and concentrated under reduced pressure. The concentrate was purified by silica gel column chromatography (eluent: PE/EtOAc=10/1) to afford the title compound as a light brown solid (1.22 g, 85%) which was used directly in the next step.

Step 4: tert-butyl (2-(4-hydroxy-1-methyl-1H-indol-3-yl)ethyl)(2-methoxybenzyl)carbamate

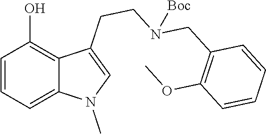 [SMILES: COc1ccccc1CN(CCc1cn(C)c2cccc(O)c12)C(=O)OC(C)(C)C]

A mixture of tert-butyl (2-(4-(benzyloxy)-1-methyl-1H-indol-3-yl)ethyl)(2-ethoxybenzyl)carbamate (512 mg, 1.02 mmol), 10% Pd/C (200 mg) and 10% Pd(OH)₂/C (200 mg) in methanol (15 mL) was stirred under hydrogen atmosphere. After the starting material was consumed, the suspension was filtered. The filtrate was concentrated and used directly in the next step.

Step 5: 3-(2-((2-methoxybenzyl)amino)ethyl)-1-methyl-1H-indol-4-ol hydrochloride

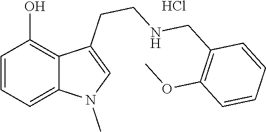 [SMILES: Cl ; COc1ccccc1CNCCc1cn(C)c2cccc(O)c12]

A solution of tert-butyl (2-(4-hydroxy-1-methyl-1H-indol-3-yl)ethyl)(2-methoxybenzyl)carbamate (415 mg, 1.0 mmol) in 2M HCl/EtOH (15 mL) was stirred at room temperature overnight. The solvent was evaporated under reduced pressure. The residue was partitioned between DCM and aq. Na₂CO₃. The organic phase was washed with brine, dried over anhydrous Na₂SO₄ and concentrated under reduced pressure. The concentrate was purified by silica gel column chromatography (eluent: DCM/MeOH/aq. NH₃=20/1/0.5) to afford the free base as a light blue solid (222 mg). The solid was dissolved in DCM (4 mL) at room temperature and 1M ethanolic HCl (1.0 mL, 1.0 mmol) was added. The resulting solution was stirred at room temperature for 30 min, and then concentrated under reduced pressure. The concentrate was triturated with ether to afford the title product as a white solid (233 mg, 34%). MS (ESI) calcd for C₁₉H₂₂N₂O₂, Exact Mass: 310.2; found: 311.3 [M+1].¹H NMR (400 MHz, CD₃OD) δ 7.43 (t, J=8.0 Hz, 1H), 7.32 (d, J=7.6 Hz, 1H), 7.04-6.95 (m, 4H), 6.87 (d, J=8.0 Hz, 1H), 6.42 (d, J=7.6 Hz, 1H), 4.21 (s, 2H), 3.74 (s, 3H), 3.63 (s, 3H), 3.39 (t, J=6.8 Hz, 2H), 3.25 (t, J=7.2 Hz, 2H).

Example 25: 3-(2-((2-methoxybenzyl) (methyl)amino)ethyl)-1-methyl-1H-indol-4-ol hydrochloride

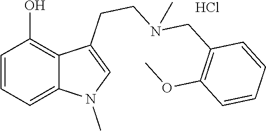 [SMILES: COc1ccccc1CN(C)CCc1cn(C)c2cccc(O)c12 ; Cl]

Step 1: 2-(4-(benzyloxy)-1-methyl-1H-indol-3-yl)-N-(2-methoxybenzyl)-N-methylethan-1-amine

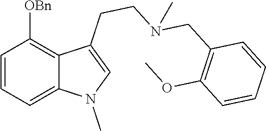 [SMILES: COc1ccccc1CN(C)CCc1cn(C)c2cccc(OCc3ccccc3)c12]

To a suspension of LiAlH₄ (162 mg, 4.26 mmol) in dried THF (7 mL) under ice-water bath and introgen atmosphere was added a solution of tert-butyl (2-(4-(benzyloxy)-1-methyl-1H-indol-3-yl)ethyl)(2-methoxybenzyl)carbamate (710 mg, 1.42 mmol) in anhydrous THF. The resulting mixture was heated at 80° C. with stirring overnight. The reaction mixture was cautiously quenched with ice-water under ice bath cooling. The mixture was filtered through celite, and the filter cake was rinsed several times with THF. The combined organic phases were dried over sodium sulfate, and concentrated under reduced pressure. The crude product residue was used directly in the next step.

Step 2: 3-(2-((2-methoxybenzyl)(methyl)amino)ethyl)-1-methyl-1H-indol-4-ol hydrochloride

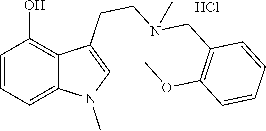 [SMILES: COc1ccccc1CN(C)CCc1cn(C)c2cccc(O)c12 ; Cl]

A mixture of the above 2-(4-(benzyloxy)-1-methyl-1H-indol-3-yl)-N-(2-methoxybenzyl)-N-methylethan-1-amine (600 mg), 10% Pd/C (150 mg) and 10% Pd(OH)₂/C (150 mg) in methanol (15 mL) was stirred under hydrogen atmosphere. After the starting material was consumed, the suspension was filtered. The filtrate was concentrated, purified by silica gel column chromatography (eluent: DCM/MeOH/aq. NH₃=30/1/0.5) to afford a gray oil (67 mg). The oil was dissolved in DCM (1 mL) at room temperature and 1M ethanolic HCl (0.3 mL, 0.3 mmol) was added. The resulting solution was stirred at room temperature for 30 min and concentrated under reduced pressure. The concentrate was triturated with ether to afford the title product as an off-white solid (52 mg, 9%). MS (ESI) calcd for C₂₀H₂₄N₂O₂, Exact Mass: 324.2; found: 325.3 [M+1]. ¹H NMR (400 MHz, CD₃OD) δ 7.34 (t, J=8.0 Hz, 1H), 7.24 (d, J=7.6 Hz, 1H), 6.93-6.82 (m, 4H), 6.74 (d, J=8.4 Hz, 1H), 6.27 (d, J=7.6 Hz, 1H), 4.37 (d, J=13.2 Hz, 1H), 4.16 (d, J=12.8 Hz, 1H), 3.61 (s, 3H), 3.68-3.45 (m, 5H), 3.34-3.27 (m, 1H), 3.21-3.17 (m, 1H), 2.81 (s, 3H).

### 5-HT₂ Gq-Dissociation BRET assays

To measure 5-HT₂ receptor-mediated Gq activation via Gq/γ1 dissociation as measured by BRET², HEK293T cells were subcultured in DMEM supplemented with 10% dialyzed FBS and were co-transfected in a 1:1:1:1 ratio with RLuc8-fused human Gαq (Gαq-RLuc8), a GFP²-fused to the C-terminus of human Gγ1(Gγ1-GFP²), human Gβ1, and 5-HT₂ receptor using TransiT-2020, as described in the literature (see: McCorvy et al., Nat Struct Mol Biol. 2018 Sep. 25, (9): 787-796; Olsen et al. Nat Chem Biol. 2020 Aug. 16, (8): 841-849). After at least 18-24 hours, transfected cells were plated in poly-lysine coated 96-well white clear bottom cell culture plates in DMEM containing 1% dialyzed FBS at a density of 25-40,000 cells in 200 μl per well and incubated overnight. The next day, media was decanted and cells were washed with 60 μL of drug buffer (1× HBSS, 20 mM HEPES, pH 7.4), then 60 μL of drug buffer was added per well. Cells were pre-incubated at in a humidified atmosphere at 37° C. before receiving drug stimulation. Drug stimulation utilized 30 pL addition of drug (3×) diluted in McCorvy buffer (1× HBSS, 20 mM HEPES, pH 7.4, supplemented with 0.3% BSA fatty acid free, 0.03% ascorbic acid) and plates were incubated at for 1 hour at 37° C. Substrate addition occurred 15 minutes before reading and utilized 10 μL of the RLuc substrate coelenterazine 400a for Gq dissociation BRET2 (Prolume/Nanolight, 5 μM final concentration). Plates were read for luminescence at 400 nm and fluorescent GFP² emission at 510 nm at 1 second per well using a Mithras LB940 (Berthold). The BRET ratios of fluorescence/luminescence were calculated per well and were plotted as a function of drug concentration using Graphpad Prism 8 (Graphpad Software Inc., San Diego, Calif.). Data were normalized to % 5-HT stimulation and analyzed using nonlinear regression “log(agonist) vs. response” to yield Emax and EC50 parameter estimates.

Pharmacology Experimental Assay, and data thereof, of compounds provided in Table 3 above are provided in Table 5 as follows:

TABLE 5
IC50 nM 5-HT2 Gq Dissociation BRET
assay (% efficacy vs 5-HT@ 100%)

| Example | 5-HT2A | 5-HT2B | 5-HT2C |
|---|---|---|---|
| 5-HT | 2.15 nM | 0.83 nM | 0.55 nM |
| Psilocin | 8.34 nM (82%) | 1.07 nM (63%) | 7.79 (95%) |
| 1 (prior art) | 86.3 nM (73%) | 31.7 nM (40%) | 9.51 nM (92%) |
| 2 | 196 nM (65%) | Not Active | 0.9 nM (79%) |
| 3 | 188 nM (48%) | Not Active | 2.7 nM (80%) |
| 4 | 1150 nM (37%) | Not Active | 3.0 nM (67%) |
| 6 | 1803 nM (18%) | Not Active | 1.61 nM (48%) |
| (prior art) | | | |
| 8 | 28 nM (29%) | Not Active | 0.12 nM (50%) |
| 9 | 4050 nM (53%) | Not Active | 3.0 nM (44%) |
| 10 | 4000 nM (50%) | Not Active | 1070 nM (60%) |
| 12 | 133 nM (66%) | 83 nM (44%) | 2.8 nM (84%) |
| 13 | 112 nM (85%) | 42.1 nM (83%) | 63.5 nM (105%) |
| 14 | 253 nM (92%) | 49.1 nM (68%) | 393 nM (98%) |
| 16 | 140 nM (55%) | 0.245 nM (19%) | 14.9 nM (54%) |
| 17 | 8400 nM (54%) | 376 nM (24%) | 833 nM (66%) |
| 18 | 241 nM (60%) | 64 nM (56%) | 31 nM (70%) |
| 21 | 497 nM (61%) | 328 nM (46%) | 15 nM (40%) |
| 22 | 4200 nM (78%) | 1420 nM (75%) | 1890 nM (82%) |
| 24 | 10 nM (93%) | 8.3 nM (56%) | 1.6 nM (115%) |
| 25 | 770 nM (39%) | 4000 nM (14%) | 2.4 nM (70%) |

Example 1. 3-[2-(Dimethylamino)ethyl]-1-methylindol-4-yl Pivalate. 3-[2-(Dimethylamino)ethyl]-1-methylindol-4-ol is dissolved in dichloromethane (3 mL/mmol) and placed under a drying tube. Triethylamine (2 equiv.) is added, followed if desired by 4-(dimethylamino)pyridine (DMAP; 0.01-0.3 equiv.) to accelerate the reaction. Pivaloyl chloride (1.5 equiv.) is added dropwise, and the mixture is stirred until the reaction is complete. Methanol (2 equiv.) is added dropwise, and the mixture is stirred for 1 h to quench residual acylating agent. After aqueous workup (dichloromethane/1M aqueous sodium hydrogen carbonate solution) and drying over sodium sulfate, the solution of the product is concentrated, and the residue is purified by column chromatography on silica gel with an ethyl acetate/hexane/triethylamine gradient. The ester is obtained by evaporation and drying under vacuum of appropriate fractions of the eluate.

Example 2. 3-[2-(Dimethylamino)ethyl]-1-methylindol-4-yl N,N-Dimethylcarbamate. This compound is prepared from 3-[2-(dimethylamino)ethyl]-1-methylindol-4-ol in analogy to Example 1, substituting N,N-dimethylcarbamyl chloride for pivaloyl chloride.

### Methods of Use

Psilocybin analogs described herein are believed to be useful in the treatment of drug resistant depression based on several clinical trials that have been reported using psilocybin itself.

A US STAR*D study has reported that more than half of all patients recruited through primary care and psychiatric clinics fail to achieve remission after first-line antidepressant treatment, and one-third were unable to experience remission after four courses of acute treatment (Rush A J, Trivedi M H, Wisniewski S R, Nierenberg A A, Stewart J W, Warden D, et al. Acute and longer-term outcomes in depressed outpatients requiring one or several treatment steps: a STAR*D report. Am. J. Psychiatry 2006; 163:1905-17).

In addition to the potential use of these analogs in the treatment of depression, other studies by third party groups of human volunteers have revealed that psilocybin can be used to treat tobacco and alcohol addiction. Moreover, in a controlled clinical environment, psilocybin was safely administered to subjects with OCD, and this drug treatment was found to lead to acute reductions in core OCD symptoms in several subjects (Moreno, F. A., Wiegand, C. B., Taitano, E. K., and Delgado, P. L. “Safety, tolerability, and efficacy of psilocybin in 9 patients with obsessive-compulsive disorder” J. Clin. Psychiatry 2006, 67, 1735-1740).

Another potential use of these analogs is in the treatment of seizure disorders, including but not limited to infantile seizure disorders such as but not limited to Dravet syndrome (Sourbon, J. et al. “Serotonergic Modulation as Effective Treatment for Dravet Syndrome in a Zebrafish Mutant Model”, ACS Chem. Neurosci. 2016, 7, 588-598).

The psilocybin analogs described herein are believed to be safer than psilocybin, given their lack at least some of the undesirable characteristics of 5-HT2B-agonist related activities.

### Methods for Administration of the Psilocybin Analogs

As contemplated herein, a therapeutically effective amount of a psilocybin analog described herein is administered to a subject in need thereof. Whether such treatment is indicated depends on the subject case, and is further subject to medical assessment (diagnosis) that takes into consideration signs, symptoms, and/or malfunctions that are present, the risks of developing particular signs, symptoms and/or malfunctions, and other factors.

As contemplated herein, a psilocybin analog described herein may be administered by any suitable route known in the art. Such routes include, but are not limited to, oral, buccal, inhalation, topical, sublingual, rectal, vaginal, intracisternal or intrathecal through lumbar puncture, transurethral, nasal, percutaneous, transdermal, and parenteral administration (including intravenous, intramuscular, subcutaneous, intracoronary, intradermal, intramammary, intraperitoneal, intraarticular, intrathecal, retrobulbar, intrapulmonary injection and/or surgical implantation at a particular site). Parenteral administration may be accomplished using a needle and syringe or using a high pressure technique.

Pharmaceutical compositions include those wherein a psilocybin analog described herein is present in a sufficient amount to be administered in an effective amount to achieve its intended purpose. The exact formulation, route of administration, and dosage is determined by a qualified medical practitioner in view of the diagnosed condition or disease. Dosage amount and interval can be adjusted individually to provide levels of a psilocybin analog described herein that is sufficient to maintain the desired therapeutic effects. It is possible that the psilocybin analog described herein may only require infrequent administration (e.g. monthly, as opposed to daily) to achieve the desired therapeutic effect.

As contemplated herein, a therapeutically effective amount of a psilocybin analog described herein adapted for use in therapy varies with the nature of the condition being treated, the length of time that activity is desired, and the age and the condition of the patient, and ultimately is determined by the attendant physician. Dosage amounts and intervals can be adjusted individually to provide plasma levels of the psilocybin analog that are sufficient to maintain the desired therapeutic effects. The desired dose conveniently may be administered in a single dose, or as multiple doses administered at appropriate intervals, for example as one, two, three, four, or more subdoses per day. Multiple doses often may be desired or required. For example, a psilocybin analog described herein may be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

As contemplated herein, The psilocybin analogs described herein may be administered in admixture with a pharmaceutical carrier selected with regard to the intended route of administration and standard pharmaceutical practice. Pharmaceutical compositions for use in accordance with the psilocybin analogs described herein are formulated in a conventional manner using one or more physiologically acceptable carriers comprising excipients and auxiliaries that facilitate processing of the present compounds.

Water is a preferred carrier when a present psilocybin analog is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The present compositions, if desired, can also contain minor amounts of wetting or emulsifying agents, or pH buffering agents.

These pharmaceutical compositions may be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of a present psilocybin analog is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a present psilocybin analog. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a present compound.

When a therapeutically effective amount of a psilocybin analog described herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains an isotonic vehicle. A psilocybin analog described herein can be infused with other fluids over a 10-30 minute span or over several hours.

The psilocybin analogs described herein may be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated.

Pharmaceutical preparations for oral use can be obtained by adding a psilocybin analog described herein to a solid excipient, with or without grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

A psilocybin analog described herein may be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a present psilocybin analog can be prepared as appropriate oily injection suspensions. Suitable lipophilic solvents or vehicles include fatty oils or synthetic fatty acid esters. Aqueous injection suspensions can contain substances which increase the viscosity of the suspension.

In some embodiments, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

A psilocybin analog described herein also may be formulated in rectal compositions, such as suppositories or retention enemas, e.g., containing conventional suppository bases. In addition to the formulations described previously, a present psilocybin analog also can be formulated as a depot preparation. Such long-acting formulations can be administered by implantation (for example, subcutaneously or intramuscularly) or by intramuscular injection. Thus, for example, a psilocybin analog described herein may be formulated with suitable polymeric or hydrophobic materials (for example, as an emulsion in an acceptable oil) or ion exchange resins.

A psilocybin analog described herein may be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The psilocybin analogs described herein also may be injected parenterally, for example, intravenously, intramuscularly, subcutaneously, or intracoronarily. For parenteral administration, the a psilocybin analog described herein may be best used in the form of a sterile aqueous solution which can contain other substances, for example, salts or monosaccharides, such as mannitol or glucose, to make the solution isotonic with blood.

### GENERAL

It is contemplated that any part of any aspect or embodiment discussed in this specification may be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

Unless defined otherwise, all technical and scientific terms used herein have the same meaning as is commonly understood by one of ordinary skill in the art to which this invention belongs. In addition, any citation of references herein is not to be construed nor considered as an admission that such references are prior art to the chemical entities described in this disclosure.

The scope of the claims should not be limited by the example embodiments set forth herein, but should be given the broadest interpretation consistent with the description as a whole.

### ACKNOWLEDGEMENT

The inventors would like to thank John McCorvy's laboratory at Medical College of Wisconsin for performing the pharmacological experimental assays on select compounds described in this disclosure.

1. A compound of Formula I or a pharmaceutically acceptable salt thereof 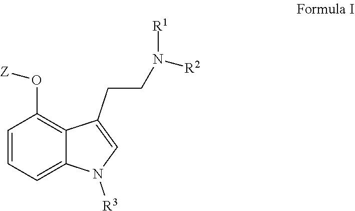
wherein: (i) R¹ is C₁-C₆ alkyl, C₂-C₆ alkenyl, C₂-C₆ alkynyl, C₃-C₆ cycloalkyl, or CH₂(C₃-C₆ cycloalkyl), substituted benzyl, halobenzyl, C₁-C₆ alkyl benzyl, C₁-C₆ alkoxy benzyl or C₁-C₆ alkyl aryl; (ii) R² is H, C₁-C₆ alkyl, C₂-C₆ alkenyl, C₂-C₆ alkynyl, C₃-C₆ cycloalkyl, CH₂(C₃-C₆ cycloalkyl), substituted benzyl, halobenzyl, C₁-C₆ alkyl benzyl, C₁-C₆ alkoxy benzyl or C₁-C₆ alkyl aryl; and (iii) R³ is C₁-C₆ alkyl, C₃-C₆ cycloalkyl, CH₂(C₃-C₆ cycloalkyl), C₃-C₆ heterocyclyl, CH₂-(C₃-C₆ heterocyclyl), 3-oxetanyl, 4-7 membered heterocyclyl, or CH₂(4-7 membered heterocyclyl); and
wherein: (A) if R¹ and R² are C₁ alkyl, then R³ is C₂-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl); (B) if R¹ and R³ are C₁ alkyl, then R² is H, C₂-C₆ alkyl, C₂-C₆ alkenyl, C₂-C₆ alkynyl, C₃-C₆ cycloalkyl, CH₂(C₃-C₆ cycloalkyl), or alkylaryl; (C) if R² and R³ are C₁ alkyl, then R¹ is H, C₂-C₆ alkyl, C₂-C₆ alkenyl, C₂-C₆ alkynyl, C₃-C₆ cycloalkyl, or CH₂(C₃-C₆ cycloalkyl); and (D) if R¹ and R² are C₁ alkyl, then R³ is C₁-C₃ alkyl, C₅-C₆ alkyl, C₃-C₆ cycloalkyl, CH₂(C₃-C₆ cycloalkyl), C₃-C₆ heterocyclyl, CH₂(C₃-C₆ heterocyclyl), 3-oxetanyl, 4-7 membered heterocyclyl, or CH₂(4-7 membered heterocyclyl);
wherein Z is H, (R⁴)(R⁵)N—C(O)—, C₁-C₆ alkyl-C(O), or C₃-C₆ cycloalkyl-C(O), wherein R⁴ and R⁵ are independently chosen from H, C₁-C₄ alkyl, and C₃-C₆ cycloalkyl, and which may be joined to form a 4-7 membered heterocyclyl group; or Z is aryl-C(O) or heteroaryl-C(O), or Z is (R⁶O)(R⁷O)P(O)—, wherein R⁶ and R⁷ are independently H or a cationic counterion of a phosphate salt form.

2. (canceled)

3. The compound as claimed in claim 1, wherein R¹ is C₁-C₄ alkyl.

4. The compound as claimed in claim 3, wherein R¹ is selected from the group consisting of methyl and ethyl.

5. The compound as claimed in claim 3, wherein R² is H, C₁-C₆ alkyl, C₂-C₆ alkenyl, C₂-C₆ alkynyl, C₃-C₆ cycloalkyl, CH₂(C₃-C₆ cycloalkyl), alkylaryl.

6-8. (canceled)

9. The compound as claimed in claim 1, wherein R³ is C₁-C₆ alkyl, C₃-C₆ cycloalkyl, CH₂(C₃-C₆ cycloalkyl), 4-7 membered heterocyclyl, or CH₂(4-7 membered heterocyclyl).

10-12. (canceled)

13. The compound as claimed in claim 1, wherein R¹ and R² are methyl and R³ is ethyl.

14. The compound as claimed in claim 1, wherein R¹ and R² are methyl and R³ is CH₂(cyclopropyl).

15. The compound as claimed in claim 1, wherein R¹ and R² are methyl and R³ is cyclopropyl.

16. The compound as claimed in claim 1, wherein R¹ and R³ are methyl and R² is CH₂(cyclopropyl).

17. The compound as claimed in claim 1, wherein R¹ and R² are each CH₃ and R³ is CD₃.

18. The compound as claimed in claim 1, wherein R¹ is substituted benzyl, R² is H, and R³ is C₁-C₆ alkyl.

19. The compound as claimed in claim 18, wherein the substituted benzyl comprises one, two, or three substituents selected from the group consisting of: C₁-C₄ alkyl, C₁-C₄ alkoxy, halo, and any combination thereof.

20. The compound as claimed in claim 18, wherein R¹ is methoxybenzyl.

21. The compound as claimed in claim 20, wherein R¹ is 2-methoxybenzyl or 3-methoxybenzyl.

22. The compound as claimed in claim 18, wherein R¹ is halobenzyl.

23. The compound as claimed in claim 22, wherein the halobenzyl is selected from the group consisting of fluorobenzyl and chlorobenzyl.

24. The compound as claimed in claim 23, wherein: (i) the fluorobenzyl is select from the group consisting of 2-fluorobenzyl and 3-fluorobenzyl; and (ii) the chlorobenzel is selected from the group consisting of 2-chlorobenzyl and 3-chlorobenzyl.

25. The compound as claimed in claim 18, wherein R³ is selected from the group consisting of methyl, ethyl, propyl and cyclopropyl.

26. The compound as claimed in claim 25, wherein R³ is methyl or ethyl.

27-29. (canceled)

30. A method of treating the symptoms of any one of depression, alcoholism, tobacco and cocaine addiction, inflammation, cluster headache, PTSD, and other CNS disorders, in a subject comprising administering to said subject an effective amount of the compound as claimed in claim 1.

31-35. (canceled)
